(19) United States
(12) Patent Application Publication
(10) Pub. No.: US 2022/0112149 A1
(43) Pub. Date: Apr. 14, 2022

(54) DRUG DESIGN METHOD ASSOCIATED WITH NATURAL PRODUCTS, PENTACYCLIC TRITERPENOID COMPOUND, PREPARATION METHOD AND APPLICATIONS THEREOF

(71) Applicant: Huazhong Agricultural University, Wuhan (CN)

(72) Inventors: Xuebo HU, Wuhan (CN); Bo ZHOU, Wuhan (CN); Le SHI, Wuhan (CN)

(21) Appl. No.: 17/394,400

(22) Filed: Aug. 4, 2021

(30) Foreign Application Priority Data
Jun. 4, 2020 (CN) 202010497221.X

(51) Int. Cl. C07C 61/125; A61P 3/04; G16C 20/50

(52) U.S. Cl. CPC C07C 61/125; C07C 2603/52; G16C 20/50; A61P 3/04

(57) ABSTRACT

A drug design method associated with natural products, includes: acquiring a molecular structure of a to-be-modified natural product with a specific biological activity; using the molecular structure as a template molecule, selecting a plurality of natural products as a reference molecule set, where the plurality of natural products has the specific biological activity and a structural similarity thereof to the template molecule is within a threshold range; and selecting one or more reference molecules from the reference molecule set, comparing the one or more reference molecules with the template molecule and determining at least one different active functional group therebetween; and constructing the at least one different active functional group on a molecular scaffold shared by the template molecule and the one or more reference molecules, thereby obtaining a modified molecular structure of the natural product with the specific biological activity.

### CROSS-REFERENCE TO RELATED APPLICATIONS

Pursuant to 35 U.S.C. § 119 and the Paris Convention Treaty, this application claims foreign priority to Chinese Patent Application No. 202010497221.X filed Jun. 4, 2020, the contents of which, including any intervening amendments thereto, are incorporated herein by reference. Inquiries from the public to applicants or assignees concerning this document or the related applications should be directed to: Matthias Scholl P. C., Attn.: Dr. Matthias Scholl Esq., 245 First Street, 18th Floor, Cambridge, Mass. 02142.

### BACKGROUND

The disclosure relates to the field of biomedicine, and more particularly, to a drug design method associated with natural products, a pentacyclic triterpenoid compound, and a preparation method and applications thereof.

Although considerable manpower and material resources have been invested in combinatorial chemistry, the only new chemical entity drug discovered through this route is sorafenib so far, which was approved by the FDA in 2005 for the treatment of renal cell carcinoma. Natural products play an important and irreplaceable role in the research and development of new drugs. Compared with synthetic compounds, natural products have more stereogenic centers, more fused, bridged or spiro ring structures, diverse molecular structures and easiness to binding to biological macromolecules.

However, many effective natural products have low natural abundance. For example, the content of paclitaxel, the first-line chemotherapy drug, is less than 0.01% in the bark of Taxus chinensis, which makes it difficult to achieve mass production. In addition, metabolic syndrome, a general term for chronic diseases, seriously endanger human health and bring huge economic burdens to the society. Studies have shown that the obesity-induced insulin resistance and type 2 diabetes are closely related to the dysfunction of the immune system. A large number of inflammatory facto, such as tumor necrosis factor-α (TNF-α), are secreted in the adipose tissues of such patients. Celastrol has anti-inflammatory and anti-obesity properties. However, Celastrol is extremely toxic, and its oral LD50 is 20.5 mg/kg. Celastrol at a dose of 4 mg/kg may lead to a lethality rate of 40% in mice, and a dose of 1 mg/kg has serious toxicity to the brains, hearts and livers of mice. In addition, Celastrol has low bio-content (0.1-0.3% of dry weight) and low bioavailability (17.06%) in host plants.

### SUMMARY

The disclosure provides a drug design method associated with natural products. The method aims to modify a natural product by screening reference molecules of the natural product with the same or similar bioactivity and higher abundance. The disclosure further provides a pentacyclic triterpenoid compound, a preparation method and application thereof, thereby solving the conventional technical problems of low abundance, environmental unfriendliness and high costs of existing natural products, as well as unsatisfied drug efficacy or toxicity that are unfavorable to druggability.

In one aspect, the disclosure provides a drug design method associated with natural products, the method comprising:
1) acquiring a molecular structure of a to-be-modified natural product with a specific biological activity;
2) using the molecular structure acquired in 1) as a template molecule, selecting a plurality of natural products as a reference molecule set, where the plurality of natural products has the specific biological activity and a structural similarity thereof to the template molecule is within a threshold range; and
3) selecting one or more reference molecules from the reference molecule set obtained in 2), comparing the one or more reference molecules with the template molecule and determining at least one different active functional group therebetween; and constructing the at least one different active functional group on a molecular scaffold shared by the template molecule and the one or more reference molecules, thereby obtaining a modified molecular structure of the natural product with the specific biological activity.

In a class of this embodiment, in 2), the threshold range of the structural similarity of the plurality of natural products with respect to the template molecule is more than 0.2, and particularly between 0.2 and 0.9.

In a class of this embodiment, a natural abundance of the plurality of natural products in the reference molecule set in corresponding host plants thereof exceeds a preset bio-content threshold; and the bio-content threshold is preferably 1%.

In a class of this embodiment, “selecting one or more reference molecules from the reference molecule set obtained in 2)” comprises: screening one or more reference molecules with the same action target and/or action channel from the reference molecule set.

“Determining at least one different active functional group” comprises: screening a plurality of active functional groups of the one or more reference molecules through a physiological model or a molecular docking model, and comparing the plurality of active functional groups with the template molecule, to determine at least one different active functional group therebetween.

According to another aspect, the disclosure provides a method for preparing a compound molecule designed according to the aforesaid drug design method, the method comprising the following steps:

S1) selecting the template molecule and a reference molecule, selecting one from the template molecule and the reference molecule which has higher bio-content in corresponding host plant species as a raw material; preferably, the reference molecule is selected as the raw material; and

S2) constructing the at least one different active functional group between the template molecule and the reference molecule on the raw material through a chemical reaction, thereby obtaining the compound molecule designed by the drug design method.

In a class of this embodiment, the specific biological activity is anti-inflammatory activity, and the to-be-modified natural product with the specific biological activity is Celastrol or corosolic acid; the reference molecule is 18-β glycyrrhetinic acid, and the preparation method comprises the following steps:

S1, selecting an 18-β glycyrrhetinic acid molecule as the raw material; and

S2, constructing a substituted hydroxyl and a conjugated enone structure on an A ring of Celastrol on the 18-β glycyrrhetic acid molecule; or constructing substituted α, β hydroxyls and a conjugated enone structure on an A ring of corosolic acid on the 18-β glycyrrhetic acid molecule.

According to another aspect herein, the disclosure provides a pentacyclic triterpenoid compound, comprising a molecular scaffold shared by a template pentacyclic triterpenoid compound and a natural product with a structural-similarity score of 0.2 or more with the template pentacyclic triterpenoid compound, and at least one different active functional group between the template pentacyclic triterpenoid compound and the natural product; the template pentacyclic triterpenoid compound is Celastrol or corosolic acid; the natural product has anti-inflammatory activity, and a natural abundance of the natural product in a host plant thereof exceeds 1%.

In a class of this embodiment, the natural product is 18-β glycyrrhetinic acid.

In a class of this embodiment, the pentacyclic triterpenoid compound has an oleanane-type triterpene-30 carboxylic acid scaffold and a 3-carbonyl-1,2-enol structure on the A ring, preferably 3,11-dicarbonyl-1,12-diene-2-hydroxy-oleanane-30 carboxylic acid, or 11-dicarbonyl-12-ene-2α,3β-dihydroxy-oleanane.

According to still another aspect, the disclosure provides a method for treatment of chronic inflammation or metabolic syndrome, the method comprising administering to a patient in need thereof the pentacyclic triterpenoid compound or a pharmaceutically acceptable salt thereof; preferably, for treatment of surgically deficient obesity or type 2 diabetes.

For the design method associated with natural products of the disclosure, through molecular structure similarity and bio-content screening, starting from the low-cost, high-abundance reference molecules and combining the structural design and chemical synthesis, the production cost of the drugs with natural products as raw materials is reduced, and the druggability of natural products is effectively improved, i.e. the toxicity is reduced or the biological activity is enhanced. Therefore, this method is of great significance to the development of drugs associated with natural products. The drug design method can be applied to the study of large natural drugs with complex structures.

Particularly, in the disclosure, the bio-content of natural product triterpene products in the host plant and the structural similarity values with Celastrol and corosolic acid compounds are used as screening conditions. Through transformation of mother nucleus structure of the lead compound, a cheap and easy-to-obtain lead compound is designed successfully to replace triterpene molecules. The active structural fragments of the lead compound are fused into cheap template molecules, and the molecular structure is modified to obtain two triterpene derivatives with significant weight loss and anti-diabetic effects. The drug research methods, such as single-target drug design and direct derivatization of lead compounds, are used to transform the structural similarity of natural product lead compounds that are not sufficiently clear in the mechanism of drug action, have significant drug effects and have complex structures.

The pentacyclic triterpenoid compound provided in the disclosure has similar physiological effects as Celastrol and corosolic acid; in addition, it is a cheap natural product derivative and is easy to prepare in large quantities. Experiments have confirmed that they have obvious physiological activities against inflammation and chronic inflammation-induced metabolic syndrome.

### BRIEF DESCRIPTION OF THE DRAWINGS

FIGS. 1A-1C show a flow chart of a drug design method associated with natural products provided in Example 1; where, FIG. 1A: a schematic diagram of flow; FIG. 1B. a two-dimensional correlation diagram of 3D structural similarity and plant host biological content; FIG. 1C. 3D molecular overlay diagram of Celastrol and glycyrrhetinic acid.

FIG. 2 shows a structural formula of the template molecule Celastrol provided in Example 1.

FIG. 3 shows the evaluation of anti-inflammatory biological activity of triterpene candidate molecules.

FIG. 4 shows a molecular structure formula of template molecule corosolic acid provided in Example 2.

FIG. 5 shows a molecular structure design based on Celastrol structure fragments and glycyrrhetinic acid scaffold structures in Example 3.

FIGS. 6A-6B show a structural identification result diagram of GA-01 provided in Example 3, where FIG. 6A is the H NMR spectrum and FIG. 6B is the C NMR spectrum.

FIGS. 7A-7B show a structural identification result diagram of GA-02 provided in Example 3, where FIG. 7A is the H NMR spectrum and FIG. 7B is the C NMR spectrum.

FIG. 8 shows a molecular structure design based on the structural fragments of corosolic acid and the scaffold structure of glycyrrhetinic acid in Example 4;

FIG. 9 shows a structural identification result diagram of GA-03 provided in Example 4, and is a H NMR spectrum.

FIGS. 10A-10C show the inhibition verification effect chart of GA-02 in Example 5, where FIG. 10A: GA-02 inhibited LPS-induced ICAM-1 expression activity. The inflammatory pathway of HMEC-1 cell was activated by 1 μg/mL LPS, to determine the effect of Celastrol and GA-02 on the expression of ICAM-1 induced by LPS. FIG. 10B: The effect of GA-02 on NF-κB signaling. The NF-κB luciferase reporter plasmid was used to transfect HEK293 cells, and the effects of Celastrol and GA-02 on the NF-κB activity induced by TNF-α were determined; FIG. 10C: qRT-PCR was used to detect mRNA relative expressions of IL-1β, TNF-α, IL-6 and MCP-1.

FIGS. 11A-11C show the effect of GA-02 provided in Example 6 on the body weights and food intakes of obese high-fat-induced obesity mice. Where, DIO mice were injected with GA-02 (4, 12, 20 mg/kg) and blank vehicle. (FIG. 11A) Daily weight change of mice, (FIG. 11B) percentage of body weight changes of mice, (FIG. 11C) Reduced body weight (g) of mice in each group after 14 days of administration. The experiments in a-c were completed in two independent groups, 6 obese model mice in each cage.

FIGS. 12A-12C show the effect of GA-02 provided by Example 6 on the body fat content of obese high-fat-induced obesity mice, where, FIG. 12A: changes in fat and lean body mass of obese model mice after administered with low, medium and high doses (4,12,20 mg/kg); FIG. 12B. Changes in body fats of obese model mice after administered with low, medium and high doses (4,12,20 mg/kg); FIG. 12C. NMR imaging of body fats of obese model mice after administered with low, medium and high doses (4,12,20 mg/kg).

FIGS. 13A-13F show the changes in body shape, liver, epididymal fat, and fasting blood glucose of obese mice before and after GA-02 treatment provided in Example 6, where, FIG. 13A: Changes in the back shape of obese model mice after administered with low, medium and high doses (4,12,20 mg/kg); FIG. 13B. changes in abdomen shape of obese model mice after administered with low, medium and high doses (4,12,20 mg/kg); FIGS. 13C-13E: changes in shapes and masses of liver and epididymal fats of obese model mice after administered with low, medium and high doses (4,12,20 mg/kg); (FIG. 13F) fasting blood glucose of obese model mice after administered with low, medium and high doses (4,12,20 mg/kg).

FIGS. 14A-14D show the results of the average daily food intakes of high-fat-induced obese mice with GA-02 in the first three days provided by Example 6, where FIG. 14A: the average daily food intake of obese mice in the first three days after GA-02 administration; FIG. 14B: the daily weight change of obese mice in the first three days after GA-02 administration; FIG. 14C: percentage of decreased body weight per day for obese mice in the first three days after GA-02 administration; FIG. 14D: Average daily food intakes of obese mice in the first and second weeks after GA-02 administration.

FIGS. 15A-15H show an anatomical diagram of the main visceral organs of the high-fat-induced obese mice before and after GA-02 treatment provided in Example 6; where, FIG. 15A: the heart and attached fats of the obese model mice; FIG. 15B: the livers of the obese model mice; FIG. 15C: attached fats of kidney and around the kidneys of obese model mice; FIG. 15D: abdominal fats of obese model mice; FIG. 15E: heart, kidney and abdominal cavity of obese model mice 14 days after administration of GA-02.

FIGS. 16A-16C show the oil red O staining and H&E staining sections of liver function tests of high-fat-induced obese mice before and after GA-02 treatment provided in Example 6, where, FIG. 16A: oil red O staining and H&E staining of livers of obese mice after administration of blank vehicle and high-dose GA-02 (20 mg/kg); FIG. 16B: Changes in ALT in obese mice before and after administration; FIG. 16C: Changes in AST in obese mice before and after administration (blank control group, n=4; GA-02 group, n=4).

FIGS. 17A-17D show the results of glucose tolerance and insulin sensitivity in obese mice before and after GA-02 treatment provided in Example 6, where, FIG. 17A: the blood glucose change curve at different time points in the glucose tolerance test in the vehicle control group and GA-02 treatment group; FIG. 17B: AUC of blood glucose curve of glucose tolerance test in the vehicle control group and GA-02 treatment group; FIG. 17C: the blood glucose change curve at different time points in the insulin sensitivity test in the vehicle control group and GA-02 treatment group; FIG. 17D. AUC of blood glucose curve of insulin sensitivity test in the vehicle control group and GA-02 treatment group.

FIGS. 18A-18F show effect of GA-02 administration on the body weight and food intake of non-obese mice provided in Example 6. Where, (a-c) 22 g of lean mice were treated with blank vehicle and GA-02 for 14 days, (FIG. 18A) Body weight change, (FIG. 18B) body weight change (%), (FIG. 18C) average daily food intake; (n=6, blank vehicle group; n=8, GA-02 treatment group); (FIGS. 18D-18F) mice with body weight of about 28 g after fed conventional feeds for 14 weeks were administered with blank vehicle and GA-02 for 14 days; (FIG. 18D) body weight change, (FIG. 18E) body weight change (%), (FIG. 18F) average daily food intake; (n=6, blank vehicle group; n=8, GA-02 treatment group).

FIGS. 19A-19F show effect of GA-02 on the body weight and food intake of ob/ob and db/db mice in Example 19, where, FIG. 19A: Effect of intraperitoneal administration of vehicle control and GA-02 on the body weights of db/db mice for 14 consecutive days; FIG. 19B: body weight reduction percentage of db/db mice after intraperitoneal administration of vehicle control and GA-02 for 14 consecutive days; FIG. 19C: average daily food intake of db/db mice after intraperitoneal administration of vehicle control and GA-02 for 14 consecutive days; FIG. 19D: Effect on body weights of ob/ob mice after intraperitoneal administration of vehicle control and GA-02 for 14 consecutive days; FIG. 19E: body weight reduction percentage of ob/ob mice after intraperitoneal administration of vehicle control and GA-02 for 14 consecutive days; FIG. 19F: average daily food intake of ob/ob mice after intraperitoneal administration of vehicle control and GA-02 for 14 consecutive days.

FIGS. 20A-20I show effect of intragastric administration of GA-02 on the body weight and food intake of obese mice with varying degrees of obesity provided in Example 6, where, (FIGS. 20A-20C) high-fat-induced obesity mice, (FIGS. 20D-20F) mildly obese mice fed by conventional feeds, (FIGS. 20G-20I) conventional lean mice after intragastric administration of blank vehicle and GA-02 (40 mg/kg) for 14 days, FIG. 20A: body weights of obese mice (g), FIG. 20B: body weight change percentage (%) of obese mice, FIG. 20C: average daily food intake (g), (5 mice/group); FIG. 20D: body weights of mildly obese mice (g), FIG. 20E: body weight change percentage (%) of mildly obese mice; FIG. 20F: average daily food intake (g), (6 mice/group); FIG. 20G: body weights of lean mice (g), FIG. 20H: body weight change percentage (%) of lean mice; i: average daily food intake (g), (6 mice/group).

FIGS. 21A-21C show the effect of GA-03 on the body weight and food intake of high-fat-induced obese mice provided in Example 7. Where, intraperitoneal injection of GA-03 (2,4,8 mg/kg) and blank vehicle for 21 days in DIO mice. (FIG. 21A) daily body weights of mice, (FIG. 21B) body weight change percentage (%) of mice, (FIG. 21C) reduced body weight (g) of mice after 14 days of administration. FIGS. 21A-21C: completed by two independent groups, 6 obese model mice per cage.

FIGS. 22A-22D show the effect of GA-03 on the body fat content of high-fat-induced obesity mice provided in Example 7, where, FIG. 22A: the average daily food intake of the obese mice in the first three days after GA-03 administration; FIG. 22B: the daily body weight change of obese mice in the first three days after GA-03 administration; FIG. 22C: daily body weight reduction percentage of obese mice in the first three days after GA-03 administration; FIG. 22D: the average daily food intake of the obese mice in the first, second and third weeks after GA-03 administration.

FIGS. 23A-23F show the changes in body shape, liver, epididymal fat, and fasting blood glucose of obese mice before and after GA-03 treatment provided in Example 7, where, FIG. 23A: Changes in the back shape of obese model mice after administered with low, medium and high doses (2,4,8 mg/kg); FIG. 23B. changes in abdomen shape of obese model mice after administered with low, medium and high doses (2,4,8 mg/kg); FIGS. 23C-23E: Changes in shapes and masses of liver and epididymal fats of obese model mice after administered with low, medium and high doses (2,4,8 mg/kg); (FIG. 23F) fasting blood glucose of obese model mice after administered with low, medium and high doses (2,4,8 mg/kg).

FIGS. 24A-24C show the oil red O staining and H&E staining sections of liver function tests of high-fat-induced obese mice before and after GA-03 treatment provided in Example 7, where, FIG. 24A: oil red O staining and H&E staining of livers of obese mice after administration of blank vehicle and high-dose GA-03 (8 mg/kg) for three weeks; FIG. 24B: Changes in ALT in obese mice before and after administration; FIG. 24C: Changes in AST in obese mice before and after administration (blank control group, n=5; GA-03 group, n=5).

FIGS. 25A-25D show the results of glucose tolerance and insulin sensitivity in obese mice before and after GA-03 treatment provided in Example 7, where, FIG. 25A: the blood glucose change curve at different time points in the glucose tolerance test in the vehicle control group and GA-03 treatment group; FIG. 25B: AUC of blood glucose curve of glucose tolerance test in the vehicle control group and GA-03 treatment group; FIG. 25C: the blood glucose change curve at different time points in the insulin sensitivity test in the vehicle control group and GA-03 treatment group; FIG. 25D. AUC of blood glucose curve of insulin sensitivity test in the vehicle control group and GA-03 treatment group;

FIGS. 26A-26D show the effect of GA-03 on the body weight and food intake of lean mice and mildly obese mice provided in Example 7. Where, intraperitoneal injection of GA-03 (2,4,8 mg/kg) and blank vehicle for 21 days in lean mice fed with 20 g and 30 g ordinary feeds, (FIG. 26A) body weights of 20 g lean mice every day, (FIG. 26B) average food intake change of 20 g lean mice, (FIG. 26C) body weights of 30 g lean mice every day, (FIG. 26D) average food intake change of 30 g lean mice. a-d: completed by two independent groups, 6 mice per cage.

FIGS. 27A-27D show the results of glucose tolerance and insulin sensitivity in lean mice before and after GA-03 treatment provided in Example 7, where the glucose tolerance test and insulin sensitivity test were conducted in 20 g lean mice after intraperitoneal injection of GA-03 (2,4,8 mg/kg) and blank vehicle for 21 days. FIG. 27A: the blood glucose change curve at different time points in the glucose tolerance test in the vehicle control group and GA-03 treatment group; FIG. 27B: AUC of blood glucose curve of glucose tolerance test in the vehicle control group and GA-03 treatment group; FIG. 27C: the blood glucose change curve at different time points in the insulin sensitivity test in the vehicle control group and GA-03 treatment group; FIG. 27D. AUC of blood glucose curve of insulin sensitivity test in the vehicle control group and GA-03 treatment group.

### DETAILED DESCRIPTION

To further illustrate, embodiments detailing a drug design method associated with natural products are described below. It should be noted that the following embodiments are intended to describe and not to limit the disclosure.

The disclosure provides a drug design method associated with natural products, the method comprising the following steps:

(1) acquiring a molecular structure of a to-be-modified natural product with a specific biological activity;

(2) using the molecular structure acquired in 1) as a template molecule, selecting a plurality of natural products as a reference molecule set, where the plurality of natural products has the specific biological activity and a structural similarity thereof to the template molecule is within a threshold range.

The threshold range is within the range of structural-similarity score with the template molecule of more than 0.2, preferably within the range of less than 0.9.

Preferably, the natural abundance of the natural product in the reference molecule set in a corresponding host plant exceeds a preset bio-content threshold; the bio-content threshold is preferably 1%.

(3) Selecting one or more reference molecules from the reference molecule set obtained in 2), comparing the one or more reference molecules with the template molecule and determining at least one different active functional group therebetween; and constructing the at least one different active functional group on a molecular scaffold shared by the template molecule and the one or more reference molecules, thereby obtaining a modified molecular structure of the natural product with the specific biological activity.

“Selecting one or more reference molecules from the reference molecule set obtained in 2)” comprises: screening one or more reference molecules with the same action target and/or action channel from the reference molecule set obtained in 2);

“Determining at least one different active functional group” comprises: screening a plurality of active functional groups of the one or more reference molecules through a physiological model or a molecular docking model, and comparing the plurality of active functional groups with the template molecule, to determine at least one different active functional group therebetween.

The compound molecule designed by the drug design method can be prepared according to the following method:

S1, selecting the template molecule and a reference molecule, selecting one from the template molecule and the reference molecule which has higher bio-content in corresponding host plant species as a raw material; generally, the template molecule has excellent biological activity, but it is likely to be limited by its biological content, causing poor druggability; while the reference molecule is obtained through molecular screening, and its cost of natural source is controllable, so the compound of the reference molecule is selected as the raw material;

S2, constructing the at least one different active functional group between the template molecule and the reference molecule on the raw material through a chemical reaction, thereby obtaining the compound molecule designed by the drug design method.

According to the method provided in the disclosure, for the metabolic syndrome caused by chronic inflammation, such as obesity, the following compounds with anti-inflammatory and therapeutic effects on metabolic syndrome including obesity have been designed and screened.

The disclosure provides a pentacyclic triterpenoid compound, comprising a molecular scaffold shared by a template pentacyclic triterpenoid compound and a natural product with a structural-similarity score of 0.2 or more with the template pentacyclic triterpenoid compound, and at least one different active functional group between the template pentacyclic triterpenoid compound and the natural product; the template pentacyclic triterpenoid compound is Celastrol or corosolic acid; the natural product has anti-inflammatory activity, and a natural abundance of the natural product in a host plant thereof exceeds 1%.

More preferably, the reference molecule is 18-β glycyrrhetinic acid;

The pentacyclic triterpenoid compound has an oleanane-type triterpene-30 carboxylic acid scaffold and a 3-carbonyl-1,2-enol structure on the A ring.

The pentacyclic triterpenoid compound is 3,11-dicarbonyl-1,12-diene-2-hydroxy-oleanane-30 carboxylic acid, or 11-dicarbonyl-12-ene-2α,3β-dihydroxy-oleanane.

The pentacyclic triterpene compound provided in the disclosure is prepared according to the following method:

S1, selecting an 18-β glycyrrhetinic acid molecule as the raw material;

S2, constructing at least one different active functional group between the template molecule and the reference molecule on the raw material through a chemical reaction, thereby obtaining the compound molecule designed by the drug design method; for example, constructing the different active functional group (a substituted hydroxyl and a conjugated enone structure on the A ring) between Celastrol and 18-β glycyrrhetinic acid on the 18-β glycyrrhetic acid molecule; or constructing the different active functional group (substituted α, β hydroxyls and a conjugated enone structure on an A ring of corosolic acid) between corosolic acid and 18-β glycyrrhetinic acid on the 18-β glycyrrhetic acid molecule.

Specifically, constructing the different active functional group (a substituted hydroxyl and a conjugated enone structure on the A ring) between Celastrol and 18-β glycyrrhetinic acid on the 18-β glycyrrhetic acid molecule, comprises:

S2-1, constructing a carbonyl group to 18-β glycyrrhetinic acid through Jones oxidation reaction, to obtain 3,11-dicarbonyl-12-ene-oleanane-30 carboxylic acid; and

S2-2, constructing a 2-enol structure onto 3,11-dicarbonyl-12-ene-oleanane-30 carboxylic acid through oxygen oxidation reaction mediated by tert-butanol/potassium tert-butoxide, to obtain 3,11-dicarbonyl-1,12-diene-2-hydroxy-oleanane-30 carboxylic acid.

Specifically, constructing the different active functional group (substituted α, β hydroxyls and a conjugated enone structure on the A ring) between corosolic acid and 18-β glycyrrhetinic acid on the 18-β glycyrrhetic acid molecule, comprises:

S2-1, constructing a carbonyl group to 18-β glycyrrhetinic acid through Jones oxidation reaction, to obtain 3,11-dicarbonyl-12-ene-oleanane-30 carboxylic acid;

S2-2, constructing a 2-enol structure onto 3,11-dicarbonyl-12-ene-oleanane-30 carboxylic acid through oxygen oxidation reaction mediated by tert-butanol/potassium tert-butoxide to obtain 3,11-dicarbonyl-1,12-diene-2-hydroxy-oleanane-30 carboxylic acid; and

S2-3, constructing 2-α-3β-dihydroxy to 3,11-dicarbonyl-1,12-diene-2-hydroxy-oleanane-30 carboxylic acid through Jones oxidation, potassium tert-butoxide\tert-butanol\oxygen oxidation, and sodium borohydride reduction, to obtain 11-dicarbonyl-12-ene-2α,3β-dihydroxy-oleanane.

Through experiments, the pentacyclic triterpenoid compound or its pharmaceutically acceptable salt (such as NH₄⁺, Na⁺, K⁺ or Mg²⁺ salts;) provided by the disclosure have been confirmed to have the activity of inhibiting the expression of ICAM-1 in human microvascular endothelial cells, and therefore exhibit a good effect on suppressing chronic inflammations and an obvious therapeutic effect on metabolic syndrome including obesity and type 2 diabetes caused by overnutrition.

### EXAMPLE 1

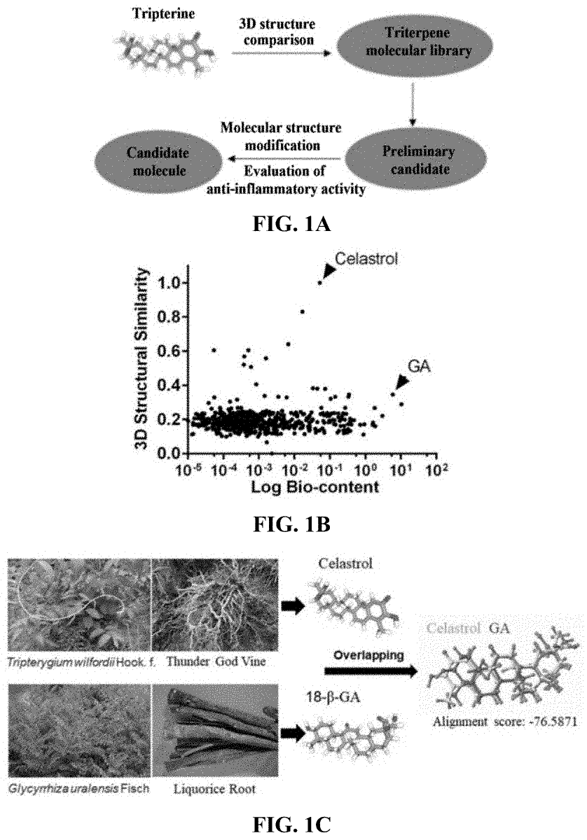

Designing anti-inflammatory active molecules using the design method associated with natural products provided in the disclosure as shown in FIGS. 1A-1C, comprises the following steps:

(1) Selecting Celastrol as the natural product molecule to be modified, that is, the template molecule;

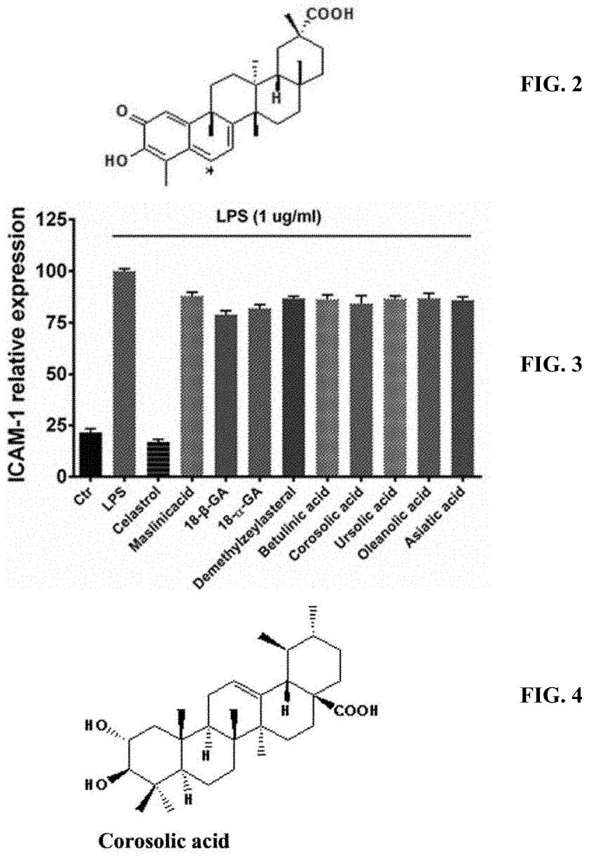

Celastrol is an active pentacyclic triterpene natural product isolated from the rhizome of Tripterygium wilfordii Hook. f. in the Celastraceae, and its structure is shown in FIG. 2. Although Celastrol has potent anti-inflammatory, anti-tumor activity and obesity treatment effects, some poor pharmacological properties and low abundance will seriously hinder its druggability. Celastrol is not a drug molecule with excellent drug properties, but it is a good lead molecule in the pharmaceutical research.

(2) Screening natural triterpene molecules with similar scaffold structures from the natural product database by using Celastrol's molecular structure as a template molecule, searching its bio-content in the host plants from the original research literature of related natural products, and recording the molecular structure, CAS number, physical and chemical properties, plant sources and original documents. The obtained information is completely sorted out in the triterpenoid natural product information database.

From PubChem (https://pubchem.ncbi.nlm.nih.gov/) database, the 3D molecular structure of each natural product in the constructed triterpenoid natural product information database is exported one by one, and compared with 3D molecular structure of Celastrol one by one through the structure alignment software (Sybyl X2.0), to obtain the structural-similarity score of each molecule. By combining the natural abundance of obtained natural product in the corresponding host plant and the structural similarity score with Celastrol, a two-dimensional correlation diagram is established, and a subset of triterpene compounds with a structural similarity score higher than 0.2 and a bio-content higher than 1% is screened, and a comprehensive analysis of each natural product in the subset is performed, to screen a subset of candidate compounds, including: oleanolic acid, corosolic acid, maslinic acid, betulinic acid, ursolic acid, demethylzeylasteral, 18-α-glycyrrhizin acid, 18-β-glycyrrhetinic acid, asiatic acid.

(3) Screening candidate molecules with the strongest ICAM-1 inhibitory activity by simulating the physiological model of inflammation that human microvascular endothelial cells (HMEC-1) expressing cell adhesion molecules (ICAM-1) mediate lymphocytes to penetrate blood vessels to infiltrate the damaged tissues. The specific steps are as follows:

Pharmacological test methods and results of pentacyclic triterpene natural products inhibiting the expression activity of ICAM-1 in human microvascular endothelial cells:

1) Culture of HMEC-1 cells: HMEC-1 cells are cultured in MCDB131 medium (U.S. Sigma) that contain 10% fetal bovine serum (FBS, Zhejiang Tianhang Biotechnology Co., Ltd.), double antibody (100 U/mL penicillin and 100 μg/mL streptomycin), 1 μg/mL hydrocortisone (Sangon Biotech (Shanghai) Co., Ltd.) and 10 ng/mL human recombinant growth factor (hEGF, Sangon Biotech (Shanghai) Co., Ltd.) at 37° C., 5% CO₂, and 100% RH.

2) HMEC-1 cells are inoculated into a 24-well plate, and cultured in a cell incubator at 37° C., 5% CO₂, and 100% RH. When the cells grow to 80% abundance, different concentrations of drugs are added for 3 h (1 μM Celastrol as a positive control drug). At the end of treatment, 1 μg/mL LPS is added to activate the NF-κB signaling pathway for 12 hours. After activation, the supernatant medium is discarded, and washed three times with PBS, and 100 μL of 0.25% pancreatin (containing 0.5 mM EDTA) is added to digest in a 37° C. incubator for 3 min. When most of cells are shed, 150 μL of MCDB131 complete medium is added to terminate the digestion, shaken for 3 min, then cells are transferred to a V-shaped 96-well plate (Corning), centrifuged at 4° C., 3000 rpm for 3 min to precipitate cells. Then the supernatant medium is discarded, 100 μL of pH=7.4 buffer A (PBS+0.5% BSA+1 mM MgCl₂) is added to each well, to wash once, centrifuged at 4° C., 3000 rpm for 3 min, and the buffer A is aspirated. To each well, 100 μL of PBS solution containing 5% bovine serum albumin is added, and shaken and blocked at 120 rpm for 30 min at room temperature; after blocking, centrifuged at 4° C. and 2000 rpm for 3 min, and the supernatant is discarded, 20 μl of buffer A containing 5 μg/mL of primary antibody anti-ICAM-1 antibody LB-2, shaken and incubated at room temperature at 120 rpm for 1 h, using the buffer A without adding anti-ICAM-1 antibody LB-2 as the negative control. By detecting anti-ICAM-1 fluorescence value by flow cytometry, the expression of ICAM-1 protein in HMEC-1 cells is detected indirectly. Three duplicate wells are established for each concentration. PBS is set as a blank control, 1 uM of Celastrol is set as a positive control, to analyze the inhibitory effect of different concentrations of drugs on the expression level of ICAM-1.

As shown in FIG. 3, results show that 18-β-glycyrrhetinic acid is the natural product with the best ICAM-1 inhibitory activity, the highest bio-content and the lowest cost among the candidate triterpene natural products, but there is still a gap in the anti-inflammatory biological activity from Celastrol.

The structures of the screened natural product 18-β-glycyrrhetinic acid and Celastrol are compared and analyzed. According to the difference of molecular structure, the active functional group modification plan is made, and the target compound 3,11-dicarbonyl-1,12-diene-2-hydroxy-oleanane-30 carboxylic acid (GA-02) is designed.

The synthesis route scheme and actual synthesis method of GA-02 are described in Example 3. The anti-inflammatory activity and anti-metabolic syndrome activity is evaluated in combination with the HMEC-1 cell screening model, as described in Examples 5 and 6.

### EXAMPLE 2

The procedure is the same as that in Example 1. The template molecule is corosolic acid, and the reference molecule is 18-β glycyrrhetinic acid, with bio-content higher than 1%. The structural-similarity score between the template molecule and the reference molecule is 0.4.

Corosolic acid is a triterpene compound naturally present in Lagerstroemia grandiflora in the Rosaceae plant (FIG. 4). It is present freely in the plant or in a form of saponin. In plants, it often co-exists with its isomer maslinic acid (2α-hydroxyoleanolic acid), with similar structure and chemical properties, so it is difficult to separate them. The in vivo and in vitro experiments showed that corosolic acid can promote the absorption and utilization of glucose by cells by promoting the transport of glucose, so as to achieve its effect of lowering blood glucose. Its excitatory effect on glucose transport is similar to insulin, therefore, corosolic acid is also called plant insulin. The animal experiments showed that, corosolic acid has a significant effect of lowering blood glucose in normal rats and mice with hereditary diabetes. Its excitatory effect on glucose transport is similar to insulin, therefore, corosolic acid is also called plant insulin.

The structures of 18-β glycyrrhetinic acid and corosolic acid are compared and analyzed. The molecular modification plan is designed according to the structural difference between them, and the target compound 11-dicarbonyl-12-ene-2α, 3β-dihydroxy-oleanane (GA-03) is designed.

The synthetic route scheme and actual synthetic method of GA-03 are shown in Examples 3 and 4; the biological activity of the target molecule and corosolic acid is measured at the cellular level. Its similar pharmacology with corosolic acid is validated in the mouse models of obesity and type 2 diabetes, as described in Example 5 and 7.

### EXAMPLE 3

### Synthesis and Identification of 3,11-dicarbonyl-1,12-diene-2-hydroxy-oleanane-30 Carboxylic Acid (GA-02)

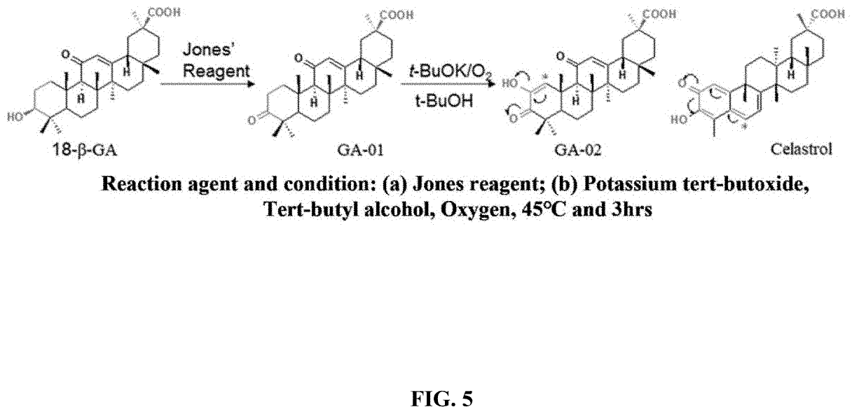

The structural modification scheme and synthetic route using 18-β glycyrrhetinic acid as a template molecule are shown in FIG. 5. The 3-en-3-hydroxy-2-one structural module of Celastrol is transferred to the A ring of glycyrrhetinic acid, with other structures of 18-β-glycyrrhetinic acid unchanged; the preparation method of the designed compound is as follows: 18-β glycyrrhetinic acid is oxidized with John's reagent to give 3,11-dicarbonyl-12-ene-oleanane-30 carboxylic acid (2); the compound (2) is oxidized by potassium tert-butoxide/oxygen in tert-butanol solvent to give 3,11-dicarbonyl-1,12-diene-2-hydroxy-oleanane-30 carboxylic acid (3); the compound (3) is recrystallized from methanol/dichloromethane mixed solvent to give pure crystals. The specific scheme is as follows:

### Synthesis of 3,11-dicarbonyl-1,12-diene-2-hydroxy-oleanane-30 carboxylic Acid

S2-1, constructing a carbonyl group to 18-β glycyrrhetinic acid through Jones oxidation reaction, to obtain 3,11-dicarbonyl-12-ene-oleanane-30 carboxylic acid;

Technical solution 1: Glycyrrhetinic acid (GA, 23.5 g 50 mmol) is added to a 500-mL round bottom flask, and 300 mL of acetone and 50 mL of dichloromethane are added to stir for 1 hour until completely dissolved, then 20 mL of freshly prepared John's reagent (2.62 g of chromium trioxide is dissolved in a small amount of water, then 2.3 mL concentrated sulfuric acid is dripped slowly, and then diluted with water to 10 mL) is added dropwise. After the complete reaction of the raw materials is monitored by TLC (developing solvent petroleum ether:ethyl acetate:acetic acid=2:1:0.01), the insoluble matter is filtered and removed, and most of the acetone is removed under a reduced pressure. The residue is added with 200 mL of distilled water, extracted with ethyl acetate 250 mL×3. The organic phase is washed with saturated brine 100 mL×2, dried over anhydrous sodium sulfate for 24 hours and then the solvent is recovered under a reduced pressure. The residual crude product is dissolved with 300 mL of ethanol by heating at 60° C., after the solution is clear, filtered while heating. The filtrate is crystallized at room temperature for 48 hours. The colorless crystal is collected by filtration and dried at 60° C. for 24 hours, to obtain 12.87 g of pure product, with a yield of 55%, MP>300° C.

Technical solution 2: Glycyrrhetinic acid (GA, 23.5 g 50 mmol) is added to a 500-mL round bottom flask, and a mixed solution of 200 mL of acetone and 200 mL of dichloromethane are added and stirred for 1 hour until completely dissolved, then 30 mL of freshly prepared John's reagent (13.1 g of chromium trioxide is dissolved in a small amount of water, then 11.5 mL concentrated sulfuric acid is dripped slowly, and then diluted with water to 50 mL) is added dropwise. After the complete reaction of the raw materials is monitored by TLC (developing solvent petroleum ether:ethyl acetate:acetic acid=2:1:0.01), the insoluble matter is filtered and removed, and most of the solvent is removed under a reduced pressure. The residue is added with 200 mL of distilled water, extracted with ethyl acetate 250 mL×3. The organic phase is washed with saturated brine 100 mL×2, dried over anhydrous sodium sulfate for 24 hours and then the solvent is recovered under a reduced pressure. The residual crude product is dissolved with 300 mL of ethanol by heating at 60° C., after the solution is clear, filtered while heating. The filtrate is crystallized at room temperature for 48 hours. The colorless crystal is collected by filtration and dried at 60° C. for 24 hours, to obtain 17.5 g of pure product, with a yield of 55%, MP>300° C.

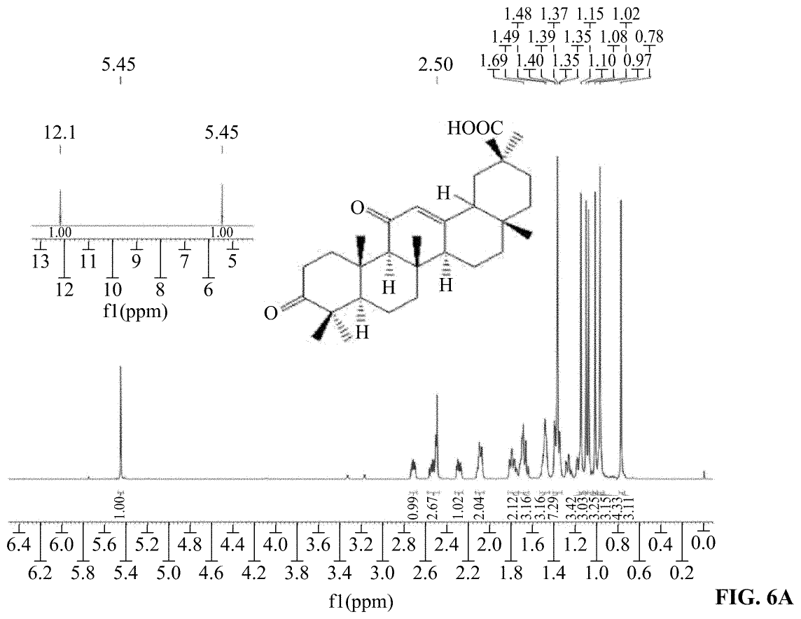

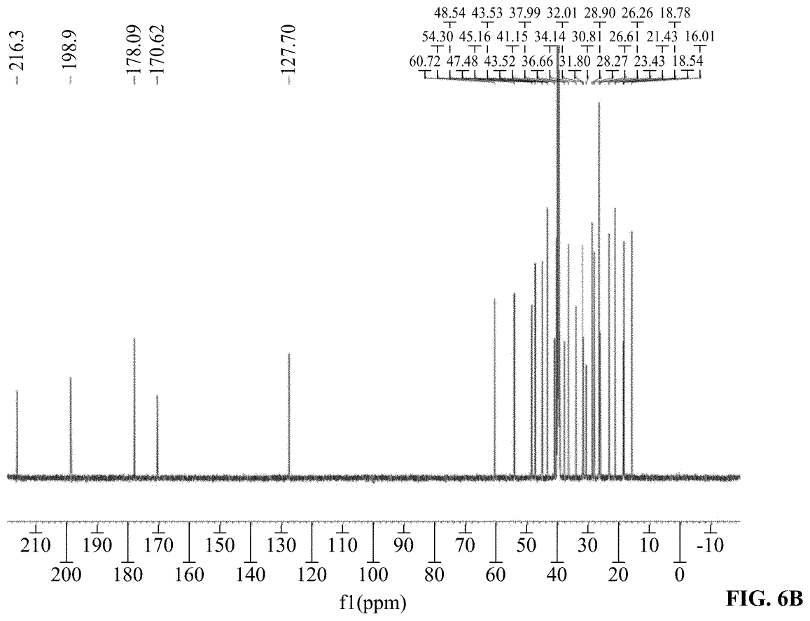

The H NMR spectrum of the product is shown in FIG. 6A, and the H and C NMR spectra are shown in FIG. 6B.

S2-2, constructing a 2-enol structure onto 3,11-dicarbonyl-12-ene-oleanane-30 carboxylic acid through oxygen oxidation reaction mediated by tert-butanol/potassium tert-butoxide, to obtain 3,11-dicarbonyl-1,12-diene-2-hydroxy-oleanane-30 carboxylic acid.

Technical solution 1: Potassium tert-butoxide (22.44 g, 20 mmol) is dissolved in 250 mL tert-butanol, stirred at 40° C. for 30 minutes. GA-01 (4.68 g, 10 mmol) is added to the reaction solution in one time, and stirred quickly for complete dissolution. The reaction carried out for 3 hours at 40° C. while stirring, and TLC (developing solvent petroleum ether:ethyl acetate:acetic acid=3:1:0.01) is used to detect the end of reaction, 4 mol/L sodium hydroxide solution is used to adjust pH to 3 to 4, and most of tertiary butanol is removed under a reduced pressure, then 200 mL of distilled water is added, extracted with ethyl acetate 250 mL×3. The organic phase is washed twice with 200 mL of saturated brine and dried over anhydrous sodium sulfate for 24 hours. The solvent is recovered under a reduced pressure to obtain the crude product, after column chromatography (petroleum ether:ethyl acetate:acetic acid=4:1:0.01), the pure components are collected and the solvent is recovered to obtain 2.65 g of pure white powder, with a yield of 55%.

Technical solution 2: GA-01 (4.68 g, 10 mmol) is dissolved in 250 mL tert-butanol, and stirred at 40° C. for 30 minutes. Potassium tert-butoxide (4.68 g, 10 mmol) is added to the reaction solution in one time, and stirred quickly for complete dissolution. The reaction is carried out for 3 hours at 40° C. while stirring, and TLC (developing solvent petroleum ether:ethyl acetate:acetic acid=3:1:0.01) is used to detect the end of reaction, 4 N sodium hydroxide solution is used to adjust pH to 3 to 4, and most of tertiary butanol is removed under a reduced pressure, then 200 mL of distilled water is added, extracted with ethyl acetate 250 mL×3. The organic phase is washed twice with 150 mL of saturated brine and dried over anhydrous sodium sulfate for 24 hours. The solvent is recovered under a reduced pressure to obtain the crude product, after column chromatography (petroleum ether:ethyl acetate:acetic acid=4:1:0.01), the pure components are collected and the solvent is recovered to obtain 2.16 g of GA-02 pure white powder, with a yield of 45%.

Technical solution 3: Potassium tert-butoxide (33.66 g, 30 mmol) is dissolved in 250 mL tert-butanol, stirred at 40° C. for 30 minutes. GA-01 (4.68 g, 10 mmol) is added to the reaction solution in one time, and stirred quickly for complete dissolution. The air is introduced to the solution, and stirred for 3 hours at 45° C. TLC (developing solvent petroleum ether:ethyl acetate:acetic acid=3:1:0.01) is used to detect the end of reaction, 4 mol/L sodium hydroxide solution is used to adjust pH to 3 to 4, and most of tertiary butanol is removed under a reduced pressure, then 200 mL of distilled water is added, extracted with ethyl acetate 250 mL×3. The organic phase is washed twice with 200 mL of saturated brine and dried over anhydrous sodium sulfate for 24 hours. The solvent is recovered under a reduced pressure to obtain a white powdery solid. The crude product is dissolved in a mixture of 300 mL methanol and 50 mL methylene chloride at 60° C. until completely dissolved. The filtrate is recovered by hot filtration and recrystallized at room temperature for 48 hours. The collected colorless crystal is dried at 60° C., to yield 3.13 g of GA-02 pure product with a yield of 65%.

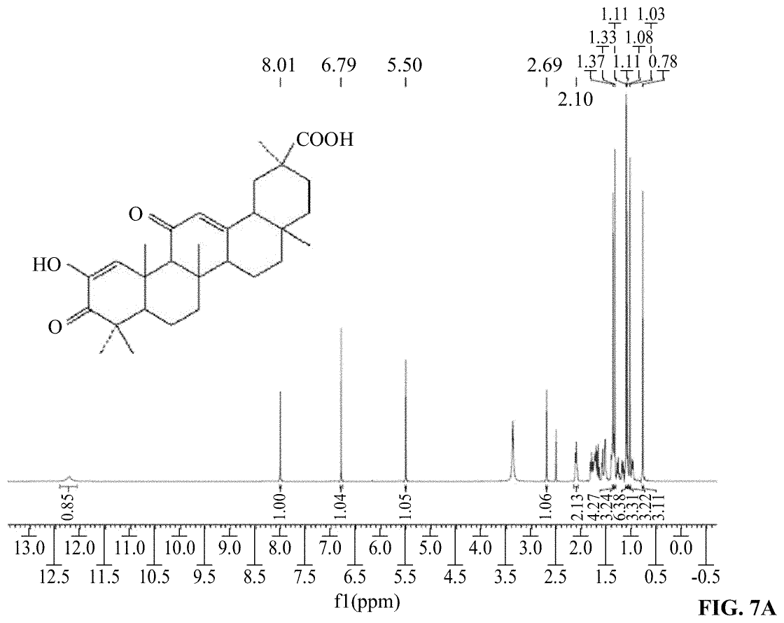

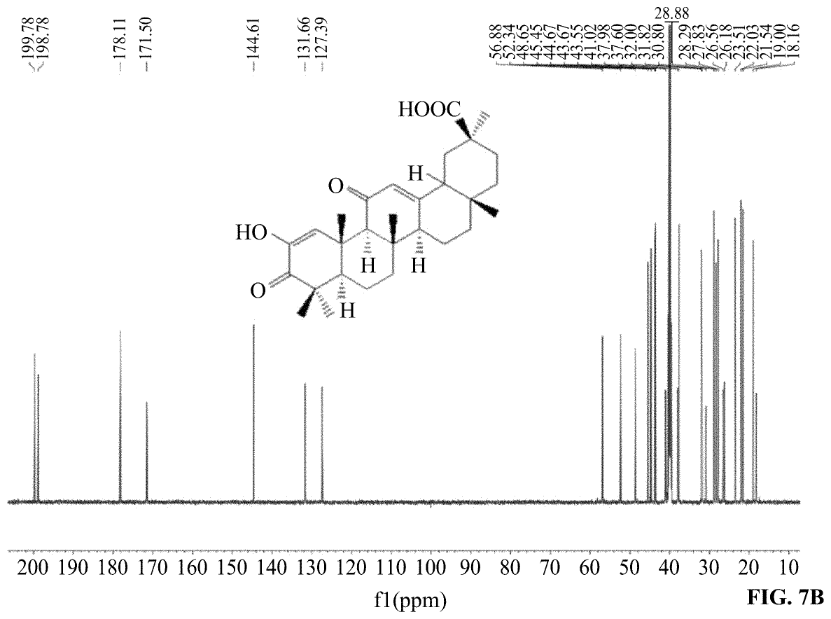

Technical solution 4: Potassium tert-butoxide (33.66 g, 30 mmol) is dissolved in 250 mL tert-butanol, stirred at 40° C. for 30 minutes. GA-01 (4.68 g, 10 mmol) is added to the reaction solution in one time, and stirred quickly for complete dissolution. The air is introduced to the solution, and stirred for 3 hours at 45° C. TLC (developing solvent petroleum ether:ethyl acetate:acetic acid=3:1:0.01) is used to detect the end of reaction, 4 mol/L sodium hydroxide solution is used to adjust pH to 3 to 4, and most of tertiary butanol is removed under a reduced pressure, then 200 mL of distilled water is added, extracted with ethyl acetate 250 mL×3. The organic phase is washed twice with 200 mL of saturated brine and dried over anhydrous sodium sulfate for 24 hours. The solvent is recovered under a reduced pressure to obtain a white powdery solid. The crude product is dissolved in a mixture of 300 mL methanol and 50 mL ammonia water at 50° C. until completely dissolved. The filtrate is recovered by hot filtration and recrystallized at room temperature for 48 hours. The collected colorless crystal is dried at 60° C., to yield 2.16 g of GA-02 pure product with a yield of 45%. The H NMR spectrum of the product is shown in FIG. 7A, and the H and C NMR spectra are shown in FIG. 7B.

### EXAMPLE 4

### Synthesis and Identification of 11-dicarbonyl-12-ene-2α,3β-dihydroxy-oleanane (GA-03)

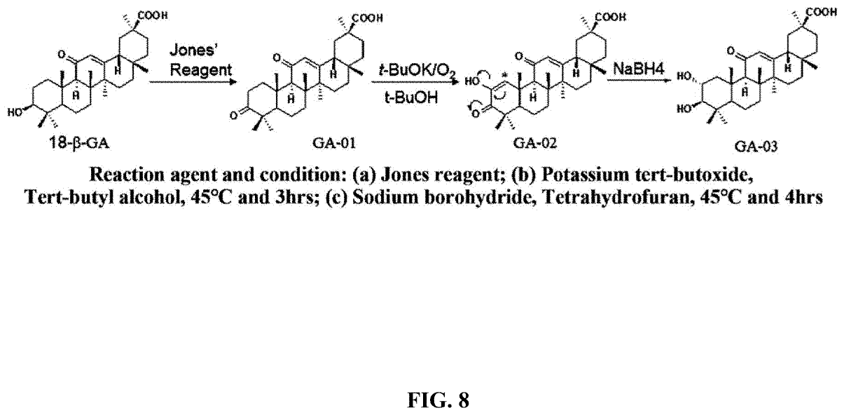

The modification scheme and synthetic route using corosolic acid as the template molecule and 18-β glycyrrhetinic acid as the reference molecule are shown in FIG. 8. The α, β hydroxyl substitutions and the conjugated enone structure on the A ring of corosolic acid are transferred to the A ring of glycyrrhetinic acid, with other structures of 18-β-glycyrrhetinic acid unchanged; the preparation method of the designed compound is as follows: 18-β glycyrrhetinic acid is oxidized with John's reagent to give 3,11-dicarbonyl-12-ene-oleanane-30 carboxylic acid (GA-01); the compound (GA-01) is oxidized by potassium tert-butoxide/oxygen in tert-butanol solvent to give 3,11-dicarbonyl-1,12-diene-2-hydroxy-oleanane-30 carboxylic acid (GA-02); the compound GA-02 is recrystallized from methanol/dichloromethane mixed solvent to give pure crystals; GA-02 is reduced by sodium borohydride in tetrahydrofuran solvent to prepare GA-03. Specific scheme of the preparation of GA-02 is the same as that in Example 3. The steps for synthesizing GA-03 using GA-02 as the raw material are as follows:

Technical solution 1: The compound GA-02 (4.82 g 10 mmol) is added into a 500-mL round-bottomed flask, 100 mL of tetrahydrofuran is added to dissolve, heated in a water bath at 42° C., then sodium borohydride (378 mg, 100 mmol) is added in batches, stirred with a magnetic rotor. During the reaction, after the completion of reaction is monitored by TLC (developing solvent petroleum ether:acetone:acetic acid=3:1:0.01 or petroleum ether:acetone:acetic acid=5:2:0.01), 50 mL of freshly prepared 2 mol/L dilute hydrochloric acid is added to neutralize the reaction, and pH is adjusted to 3 to 4, extracted with ethyl acetate 250 mL×3. 500 mL of distilled water is added to the extraction phase to wash 1 to 2 times, after separation, the aqueous phase is washed with saturated brine 250 mL×3, the organic phase is mixed, and dried over anhydrous sodium sulfate for 24 hours, then the solvent is recovered under a reduced pressure. The residual crude product is dissolved by heating with 300 mL of ethanol at 60° C., after the solution is clear, filtered while heating. The filtrate is crystallized at room temperature for 48 hours. The colorless crystal is collected by filtration and dried at 60° C. for 24 hours, to obtain 3.1 g of pure product, with a yield of 64.6%, MP>300° C.

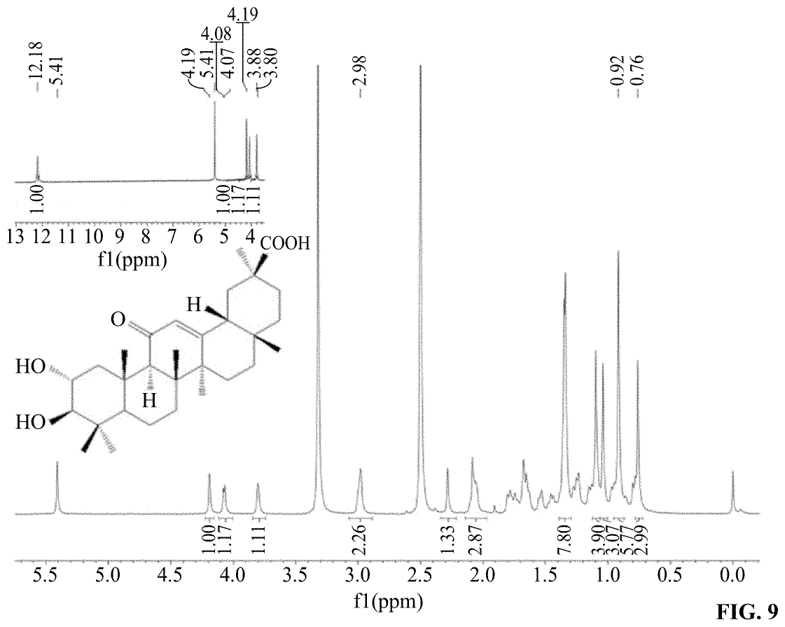

Technical solution 2: The compound GA-02 (4.82 g 10 mmol) is added into a 500-mL round-bottomed flask, 100 mL of absolute methanol is added to dissolve, and then sodium borohydride (756 mg, 200 mmol) is added in batches, stirred with a magnetic rotor. During the reaction, after the completion of reaction is monitored by TLC (developing solvent, petroleum ether:acetone:acetic acid=5:2:0.01), part of methanol is removed by evaporation. 50 mL of freshly prepared 2 mol/L dilute hydrochloric acid is added to neutralize the reaction, and pH is adjusted to 3 to 4, extracted with ethyl acetate 250 mL×3. 500 mL of distilled water is added to the extraction phase to wash 1 to 2 times, after separation, the aqueous phase is washed with saturated brine 250 mL×3, the organic phase is mixed, and dried over anhydrous sodium sulfate for 24 hours, then the solvent is recovered under a reduced pressure. The residual crude product is dissolved by heating with 300 mL of ethanol at 60° C., after the solution is clear, filtered while heating. The filtrate is crystallized at room temperature for 48 hours. The colorless crystal is collected by filtration and dried at 60° C. for 24 hours, to obtain 3.5 g of pure product, with a yield of 71.5%, MP>300° C. The H NMR spectrum of the product is shown in FIG. 9.

### EXAMPLE 5

Evaluation of Anti-Inflammatory Activity

1) Culture of HMEC-1 cells: HMEC-1 cells are cultured in MCDB131 medium (U.S. Sigma) that contain 10% fetal bovine serum (FBS, Zhejiang Tianhang Biotechnology Co., Ltd.), double antibody (100 U/ml penicillin and 100 μg/ml streptomycin), 1 μg/ml hydrocortisone (Sangon Biotech) and 10 ng/ml human recombinant growth factor (hEGF, Sangon Biotech) at 37° C., 5% CO₂, and 100% RH.

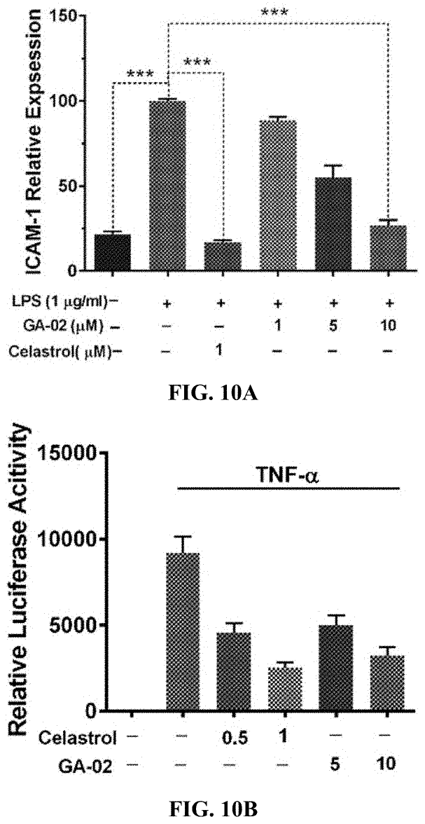

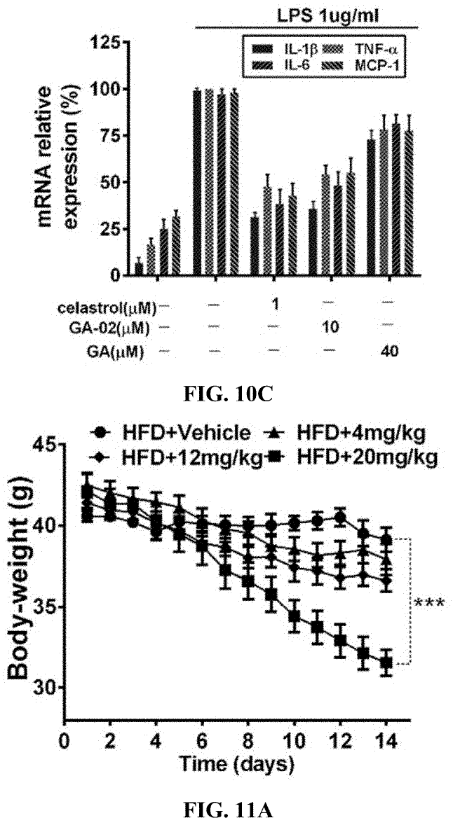

HMEC-1 cells are inoculated into a 24-well plate, and cultured in a cell incubator at 37° C., 5% CO₂, and 100% RH. When the cells grow to 80% abundance, different concentrations of drugs are added for 3 h (1 μM Celastrol as a positive control drug). At the end of treatment, 1 μg/mL LPS is added to activate the NF-κB signaling pathway for 12 hours. After activation, the supernatant medium is discarded, and washed three times with PBS, and 100 μL of 0.25% pancreatin (containing 0.5 mM EDTA) is added to digest in a 37° C. incubator for 3 min. When most of cells are shed, 150 μL of MCDB131 complete medium is added to terminate the digestion, shaken for 3 min, then cells are transferred to a V-shaped 96-well plate (Corning), centrifuged at 4° C., 3000 rpm for 3 min to precipitate cells. Then the supernatant medium is discarded, 100 μL of pH=7.4 buffer A (PBS+0.5% BSA+1 mM MgCl₂) is added to each well, to wash once, centrifuged at 4° C., 3000 rpm for 3 min, and the buffer A is aspirated. To each well, 100 μL of PBS solution containing 5% bovine serum albumin is added, and shaken and blocked at 120 rpm for 30 min at room temperature; after blocking, centrifuged at 4° C. and 2000 rpm for 3 min, and the supernatant is discarded, 20 μl of buffer A containing 5 μg/mL of primary antibody anti-ICAM-1 antibody LB-2, shaken and incubated at room temperature at 120 rpm for 1 h, using the buffer A without adding anti-ICAM-1 antibody LB-2 as the negative control. By detecting anti-ICAM-1 fluorescence value by flow cytometry, the expression of ICAM-1 protein in HMEC-1 cells is detected indirectly. Three duplicate wells are established for each concentration. PBS is set as a blank control, 1 μM of Celastrol is set as a positive control, to analyze the inhibitory effect of different concentrations of drugs on the expression level of ICAM-1. The result is shown in FIGS. 10A-10C.

According to the ICAM-1 cell expression and quantitative detection model, Celastrol at a concentration of 1 μM significantly inhibits the expression of ICAM-1 activated by LPS, and the inhibitory effect is equivalent to that of the control group. GA-02 shows a concentration-dependent ICAM-1 inhibitory activity. GA-02 at a concentration of 10 μM exhibits ICAM-1 inhibitory activity equivalent to 1 μM Celastrol.

ICAM-1 is a downstream effector molecule regulated by NF-κB signaling pathway, and NF-κB plays an important role in regulating the expression of ICAM-1 gene. Celastrol can inhibit the activation of the LPS-induced IKK/NF-κB signaling pathway. In order to validate that GA-02 functions through the IKK/NF-κB signaling pathway, we constructed an NF-κB luciferase reporter gene system to validate the inhibitory activity of Celastrol and GA-02 on the luciferase reporter gene. The experiment results showed that, 1 μg/mL LPS could activate the NF-κB pathway, and the fluorescence signal was significantly enhanced compared with that of the control group. Celastrol at a concentration of 1 μM could inhibit 70% of the fluorescence signal, and GA-02 could inhibit the fluorescence intensity in a concentration-dependent manner, and the effect of 10 μM of GA-02 is equivalent to that of 1 μM Celastrol. In addition, both Celastrol and GA-02 could down-regulate the mRNA transcription levels of inflammatory factors IL-1β, TNF-α, IL-6 and MCP-1 activated by LPS.

These experimental results showed that, Celastrol and GA-02 exerted similar anti-inflammatory activities through similar molecular structures. Although the anti-inflammatory activity of the drug was 10 times lower than that of Celastrol, it was effectively improved compared with 18β-glycyrrhetinic acid. This shows that the molecular design scheme is effective. The anti-inflammatory activity of GA-03 is evaluated using the same method, and the result shows that its anti-inflammatory activity is basically equivalent to that of GA-02 and superior to 18β-glycyrrhetinic acid.

### EXAMPLE 6

### Confirmatory Experiment on the Effect of 3,11-dicarbonyl-1,12-diene-2-hydroxy-oleanane-30 Carboxylic Acid (GA-02) Against Metabolic Syndrome

The experimental methods to validate the effect against metabolic syndrome are as follows:

1) Establishment of an Obese Mouse Model of Metabolic Syndrome and Drug Treatment Experiment

Six-week-old SPF-grade C57BL/6 mice (purchased from Hubei Provincial Center for Disease Control and Prevention) are fed in SPF animal house at a room temperature of 22° C., 4 mice per cage. Animals are fed with 60% calorie high-fat diets (purchased from Bio-medicine) for 14 consecutive weeks. Mice are free to take food and the padding is changed every two days, and the body weights and blood glucose are monitored every week. When the blood glucose is greater than 11 mmol/L and the body weight is greater than 42 g, the administration is started. The drug is dissolved in DMSO solvent and administered by intraperitoneal injection at three doses of 4, 12, and 20 mg/kg for 14 consecutive days. The injection volume is 25 μL per day. In addition, a vehicle (25 μL DMSO) and positive drug (Celastrol) control are established. The food intake and body weight changes of mice in each group are measured every day. On Days 1 and 14, the blood routine and blood biochemical parameters are monitored, and the glucose tolerance test (GTT) and insulin tolerance test (ITT) are carried out, to determine the body fat parameters of mice on Days 0 and 14. Mice in the blank group and GA-02 treatment group underwent pathological anatomy, to observe the changes in organs before and after administration. Samples are taken to prepare pathological sections.

2) Establishment of Lean Mouse Models and Drug Treatment Experiment

The animal feeding and experiment method are as follows. Six-week-old SPF-grade C57BL/6 mice (purchased from Hubei Provincial Center for Disease Control and Prevention) are fed in SPF animal house at a room temperature of 22° C., 4 mice per cage. Animals are fed with ordinary mouse feeds (purchased from) for 2 consecutive weeks. Mice are free to take food and the padding is changed every two days, and the body weights and blood glucose are monitored every week. When the body weight reaches 22 g, the administration is started. The drug is dissolved in DMSO solvent and administered by intraperitoneal injection at an injection volume of 25 μL per day. In addition, a vehicle and positive drug control are established. The food intake and body weight changes of mice in each group are measured every day. On Days 0 and 14, the blood routine and blood biochemical parameters are monitored. At the same time, the glucose tolerance test (GTT) and insulin tolerance test (ITT) are carried out.

3) Establishment of Mildly Obese Mouse Models

The animal feeding and experiment method are as follows. Six-week-old SPF-grade C57BL/6 mice (purchased from Hubei Provincial Center for Disease Control and Prevention) are fed in SPF animal house at a room temperature of 22° C., 4 mice per cage. Animals are fed with ordinary mouse feeds (purchased from) for 2 consecutive weeks. Mice are free to take food and the padding is changed every two days, and the body weights and blood glucose are monitored every week. When the body weight reaches 30 g, the administration is started. The drug is dissolved in DMSO solvent and administered by intraperitoneal injection at an injection volume of 25 μL per day. In addition, a vehicle and positive drug control are established. The food intake and body weights of mice in each group are measured every day. On Days 0 and 14, the blood routine and blood biochemical parameters are monitored.

4) Experiment in ob/ob and db/db Model Mice

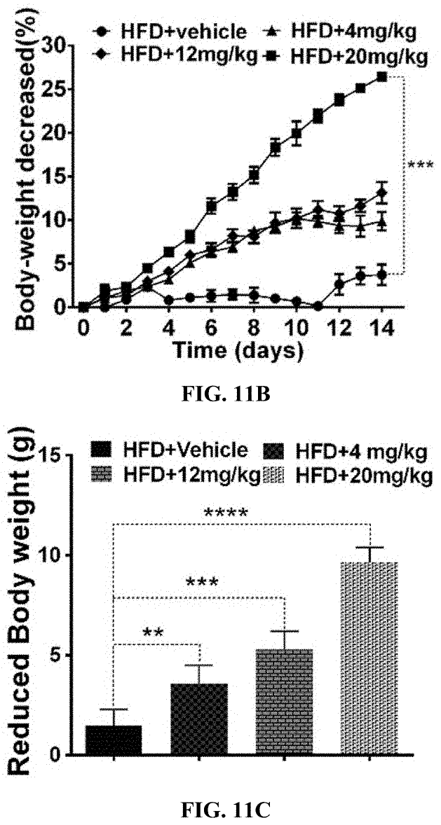

Eight-week-old male ob/ob and db/db mice are adapted to the environment under normal conditions for one week. The animals are grouped according to the body weight and blood glucose, 2 cages in each group, 5 animals in each cage. Mice are acclimatized with the vehicle for 3 days in the experimental group (25 μL DMSO/day) and are injected with drugs by intraperitoneal injection (20 mg/kg; 25 μL per injection). Mice in the control group are injected with the same volume of vehicle. The administration starts from 18:00 every day for 14 consecutive days. The body weight and food intake of mice are recorded every day. Results are shown in FIGS. 11A-11C.

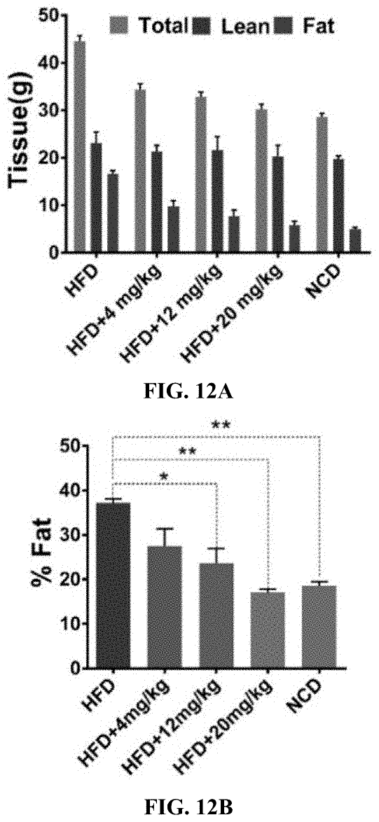

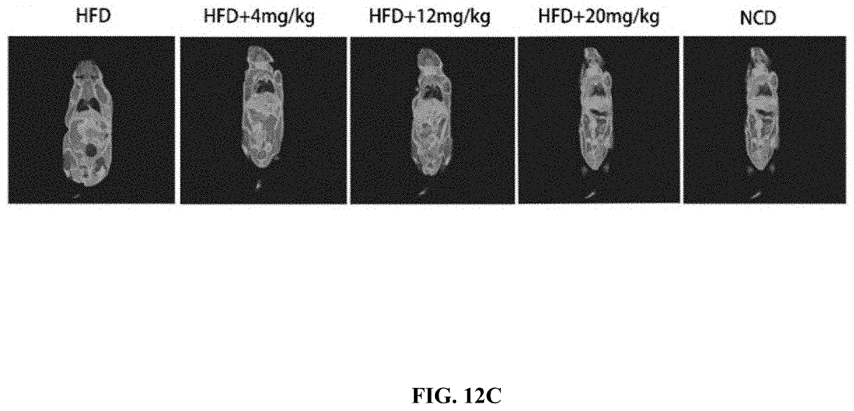

The models are constructed by feeding high-fat diets to C57BL/6 male mice for 14 weeks. Results are shown in FIGS. 12A-12C. Mice with body weight greater than 42 g and blood glucose higher than 11 mmol/L are obese model mice. Mice are grouped by blood glucose and body weight. Model mice in each group are given three doses of GA-02 (high-, medium-and low-doses, 4, 12, 20 mg/kg, 25 μL/day) by intraperitoneal injection for 14 consecutive days. Mice in the blank control group are given DMSO (25 μL/day) by intraperitoneal injection, and the food intakes and body weights are recorded every day. Two weeks after administration, the body weights in the vehicle control group did not fluctuate significantly. The body weights of the mice in the low-dose group decrease from 42.50±1.71 g to 38.46±0.95 g, reduced by 9.88±2.37%; the body weights of mice in the medium-dose group decrease from 41.41.±1.17 g to 35.45±1.15 g, reduced by 13.15±2.73%; the body weights of mice in the high-dose group decrease from 42.14±1.37 g to 30.07±1.23 g, reduced by 26.42±0.73%. The body weights of mice in each group exhibit an apparent dose-dependent reduction.

During the 14-day administration period, in the first week, the average daily food intake of mice in the vehicle control group is 2.31±0.18 g, and the average daily food intakes in the low-, medium-, and high-dose groups are 1.83±0.28 g, 1.68±0.35 g, and 0.85±0.23 g, respectively, decreased by 20.8%, 27.3% and 63.2% respectively compared to the vehicle control group; in the first week, the average daily food intake of mice in the vehicle control group is 2.47±0.13 g, and the average daily food intakes in the low-, medium-, and high-dose groups are 1.55±0.12 g, 1.35±0.18 g and 0.70±0.16 g, respectively, decreased by 37.2%, 45.3% and 68.8% respectively compared to the vehicle control group. Results show that, the body weights and average daily food intakes of obese mice exhibit a drug concentration-dependent decrease trend, and the appetite suppression and body weight reduction are most significantly in the high-dose group.

The results of NMR body fat quantitative analysis show that, the lean body masses of the mice in the low-, medium-and high-dose administration group have no significant change, while the fat contents are significantly lower than those of the obese model mice. The body fat imaging shows that the body fat of mice in the administration group is significantly lower than that of the model mice. By comparing the decrease in body weight and fat mass, it is found that most of the decrease in body weight is derived from the reduction of adipose tissue mass, and it is speculated that the decrease in body weight is caused by fat burning due to decreased food intakes.

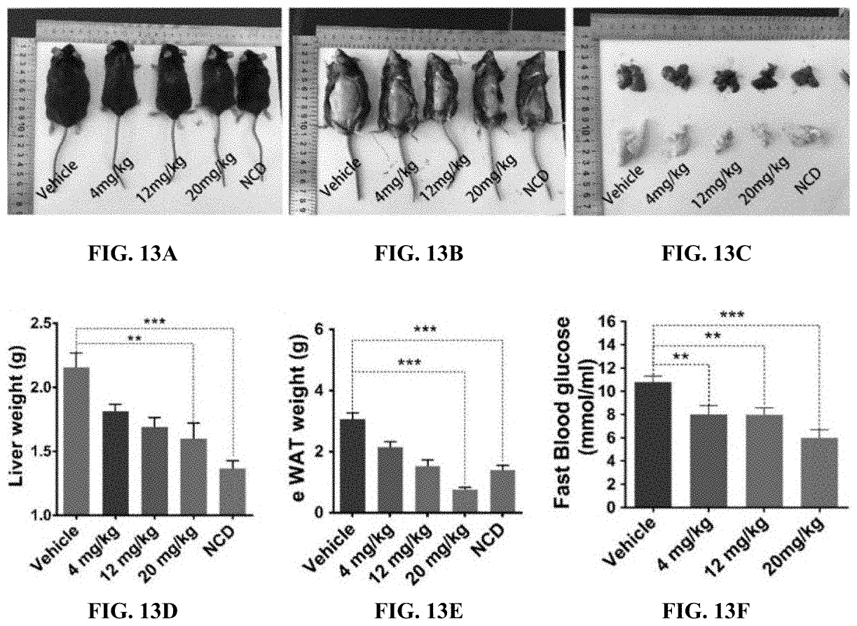

The results of anatomy of mice are shown in FIGS. 13A-13F. After administration, the mice after the administration are leaner than the blank vehicle control group. Two weeks after GA-02 administration, the fats accumulated in the abdomens of the obese mice are reduced significantly. The organ anatomy finds a lot of fat in the heart, periphery of the kidneys, epididymis and subcutaneous tissues in obese mice. The livers of the obese group show the symptoms of gray-white fatty liver withy accumulated lipid droplets. After administration, the livers become dark red. Compared to the vehicle control group, the weights of liver and epididymal fat of mice in the low-, medium-and high-dose groups decrease in a dose-dependent manner, and the fasting blood glucose level reduce to a normal level.

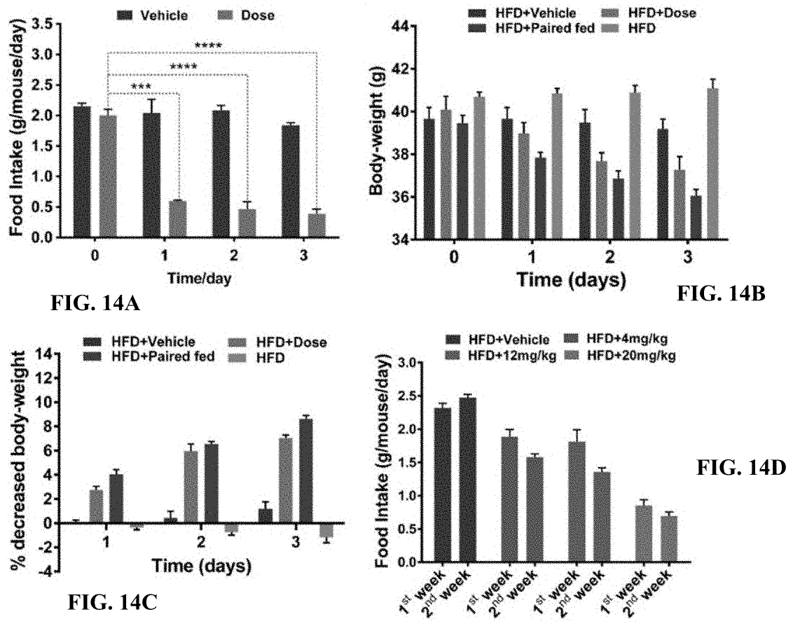

In the high-fat-induced obesity mouse models, the daily food intakes in the first three days, the first week and the second week are shown in FIGS. 14A-14D. Results show that, in the high-dose administration group, the average daily food intake on day 0 is 2.0±0.18 g, on the first day of administration, the average daily food intake decreases to 0.60±0.02 g, and the body weight reduces from 42.09±0.42 g to 40.98±0.57 g; on the second day, the average daily food intake decreases to 0.47±0.16 g, and the body weight decreases to 39.94±0.62 g; on the third day, the average daily food intake decreases to 0.39±0.11 g, and the body weight decreases to 39.23±0.71 g. The body weight decrease trend in the high-dose group is the same as that in the paired quantitative feeding group, which indirectly indicates that the decrease in body weight is derived from the reduction in the food intakes.

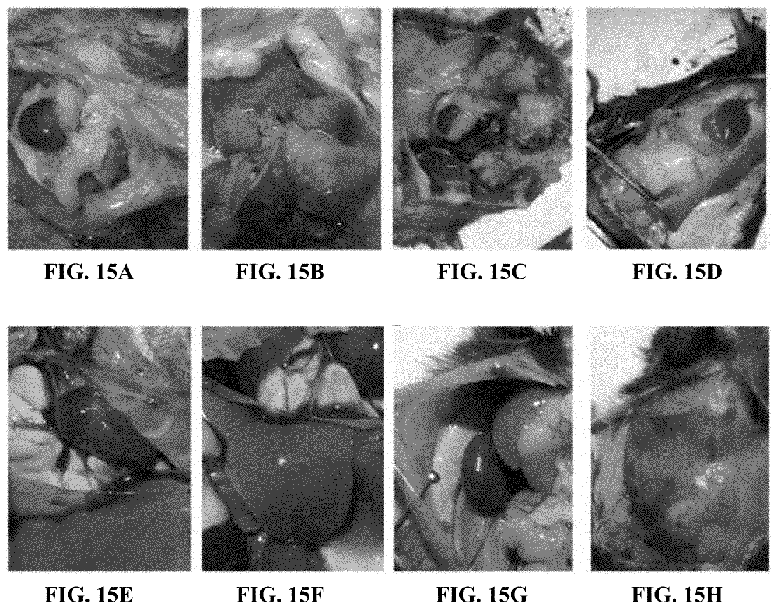

The visceral changes of obese mice before and after GA-02 administration are shown in FIGS. 15A-15H.

The dissection of mice fed with high fat diets finds that there are obvious fat accumulations in the main organs, and there are a large number of fats attached around the heart and there are obvious lipid droplets in the liver and obvious fatty liver symptoms. It is more serious that a thick layer of white fat wraps the renal tissues around the kidney tightly, and there are two large epididymal fats in the abdomen (FIGS. 15A-15D). After GA-02 administration for two weeks, the accumulated fats in the visceral tissues of the mice are completely dissipated, and it is difficult to find fats that are visible to naked eyes (FIGS. 15E-15H). In the high-dose treatment group, the heart and perirenal fats of the mice are completely dissipated, and there are no obvious fats in the subcutaneous tissues and internal organs, no obvious accumulation of lipid droplets in the liver, and no symptoms of fatty liver, indicating that the previous fatty liver is completely relieved.

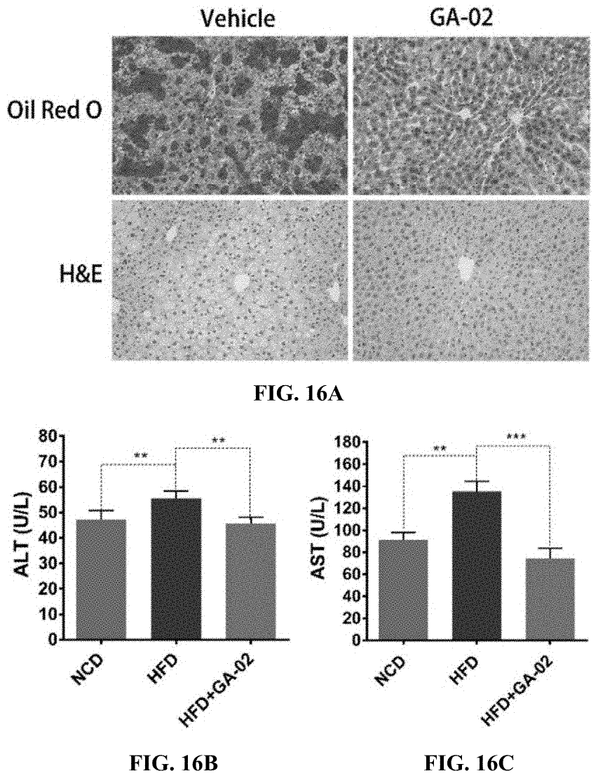

The results of liver oil red O staining and H&E staining sections are shown in FIGS. 16A-16C. As shown from the figure, there is a large number of lipid droplets in the liver in the blank control group, and the lipid droplets in the liver have completely disappeared in the high-dose GA-02 treatment group (FIG. 16A); the liver function indexes such as AST and ALT are significantly lower than those in the vehicle control group (FIGS. 16B-16C), and the results fall to normal levels; in the vehicle treatment group, the fasting blood glucose level is higher than 10 mmol/L, and the fasting blood glucose levels of mice in each group are significantly lower than those of the vehicle control group and return to the normal levels. These results show that the fatty liver symptoms caused by high-fat diets have been well relieved.

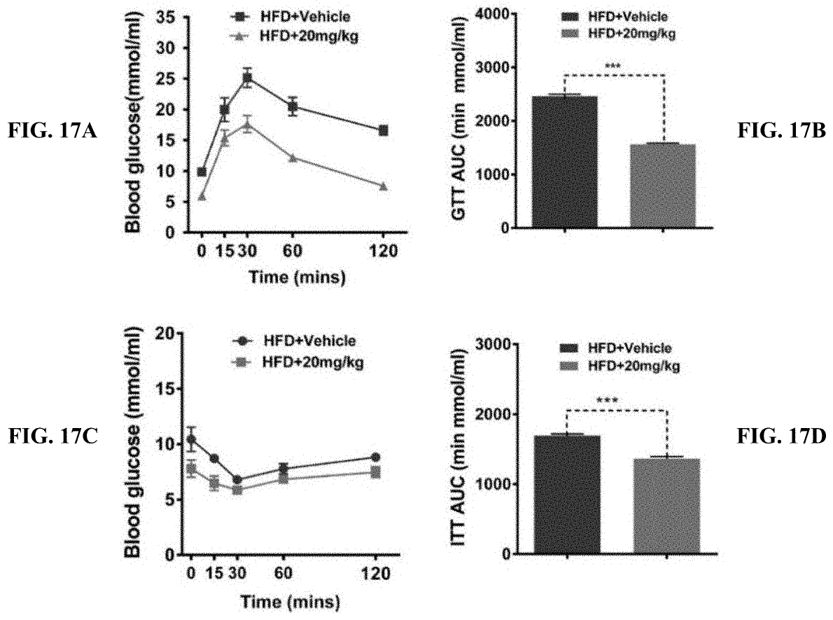

The results of glucose tolerance test (GTT) and insulin tolerance test (ITT) are shown in FIGS. 17A-17D. The diet-induced obesity is often accompanied by symptoms of diabetes such as hyperglycemia and insulin resistance. We perform GTT and ITT on the mice in the high-dose administration group. Results show the blood glucose tolerance and insulin tolerance of mice are significantly improved after GA-02 administration, and their fasting blood glucose levels are significantly reduced to normal levels, indicating that the diabetes symptoms caused by obesity have been well recovered (FIGS. 17A-17D).

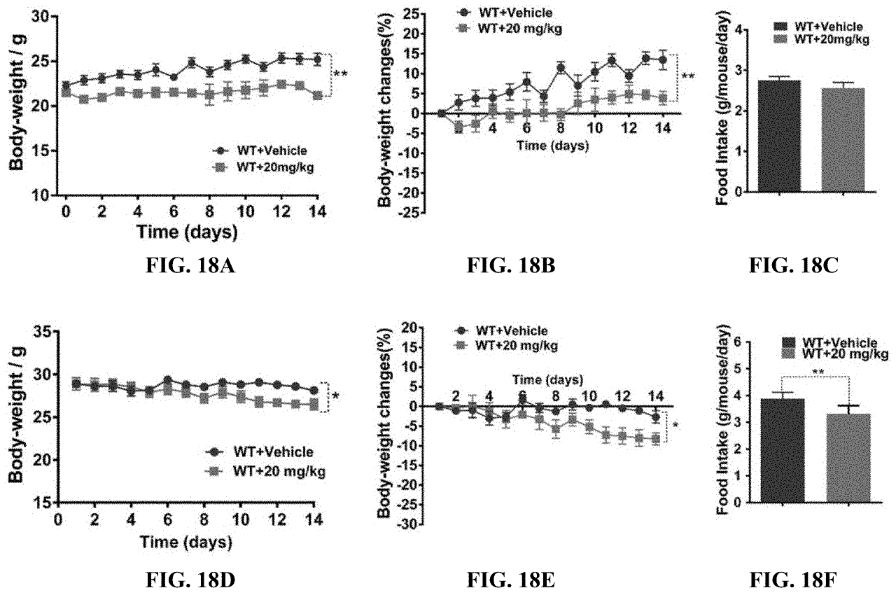

The changes in body weight and food intake of lean mice and mildly obese mice after administration are shown in FIGS. 18A-18F. If the body weights and food intakes of obese mice decrease due to toxicity, the same phenomenon will occur in the ordinary mouse group. After intraperitoneal injection of blank vehicle and high-dose GA-02 to ordinary lean mice (about 22 g), the food intakes of lean mice during the two-week administration period have no significant change compared to the vehicle control group, and the body weights show no decrease but an upward trend (FIG. 18A); in the mild obesity group (body weight of about 28 g), the vehicle has no significant effect on food intake and body weight. The food intake and body weight of the high-dose GA-02 administration group are significantly lower than those in the vehicle control group (FIGS. 18A-18F). These experimental results show that the appetite suppression and body weight reduction effects of GA-02 are not caused by toxic effects, but are positively correlated with the degree of obesity of mice.

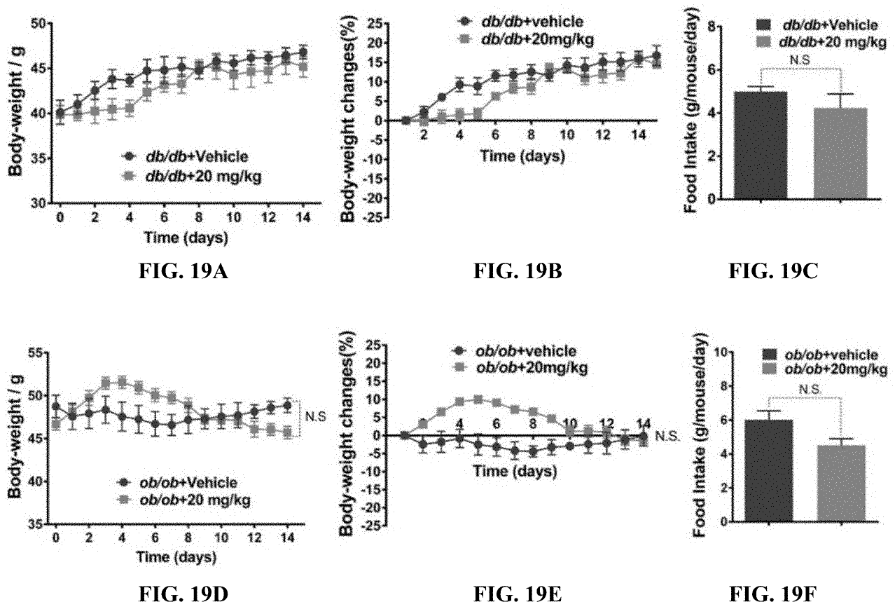

The changes in the body weight and food intake of db/db and ob/ob mice after administration are shown in FIGS. 19A-19F. After the Leptin receptor-deficient db/db mice are injected intraperitoneally with blank vehicle and GA-02 treatment for 14 days, the food intake and body weights of mice in the vehicle group have not changed significantly. The food intakes of mice in the GA-02 administration group do not change significantly compared with the vehicle control group, and their body weights show no downward trend but increase by 15% (FIGS. 19A-19C), indicating that GA-02 has no effect of suppressing appetite and reducing body weight in db/db mice. Similarly, Leptin-deficient ob/ob mice are intraperitoneally injected with blank vehicle and GA-02 for 14 days. Compared with the blank vehicle group, the food intake of mice in the GA-02 treatment group is reduced, but it is not significant, and the body weight is increased by 10% in the first five days, but later it is dropped to the initial body weight of the experiment and remains stable. Overall, the decrease in body weight is not significant (FIGS. 19D-19F). The results show that GA-02 has no effect of significantly suppressing the appetite and reducing the body weights in the ob/ob mice, and indirectly indicate that the appetite suppression and weight reduction effect of GA-02 on obese mice is related to the leptin pathway, and GA-02 has the effect of positively regulating leptin.

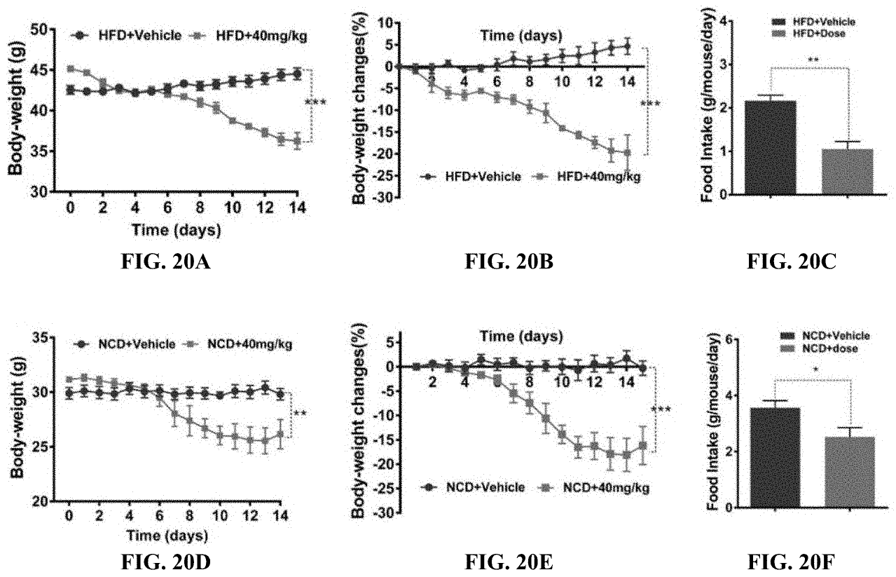

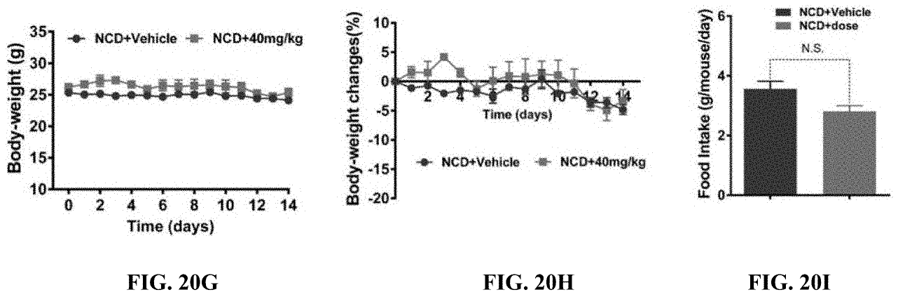

The changes in body weight and food intake of high-fat-induced obese mice and lean mice by intragastric administration are shown in FIGS. 20A-20I. The results show that the intraperitoneal injection of GA-02 has no effect on the food intake and body weight of lean mice, but has significant effect on suppressing appetite and reducing body weight of mildly obese mice, and has strong effect on suppressing appetite and reducing body weight of obese mice. Then, will the same phenomenon occur when changing the method of administration? The lean mice of about 25 g, mildly obese mice of weighing about 30 g, and obese mice of about 45 g are given blank vehicle and high-dose GA-02 (40 mg/kg) by intragastric administration, respectively.

The test results show that, intragastric administration of GA-02 has strong effect of appetite suppression and body weight reduction. After two weeks of administration, the body weight is reduced by 21%, and the average daily food intake is reduced by 50%; in the mildly obese group, the body weight is reduced by 16% and the average daily food intake is reduced by 30%; GA-02 had no significant effect on the body weight and food intake of lean mice. These test results also show that the effect of GA-02 on suppressing appetite and reducing body weight is positively related to the degree of obesity of mice (FIGS. 20A-20I).

### EXAMPLE 7

### Confirmatory Experiment on the Effect of 11-dicarbonyl-12-ene-2α,3β-dihydroxy-oleanane (GA-03) Prepared in Example 3 Against Metabolic Syndrome

1) Establishment of an Obese Mouse Model of Metabolic Syndrome

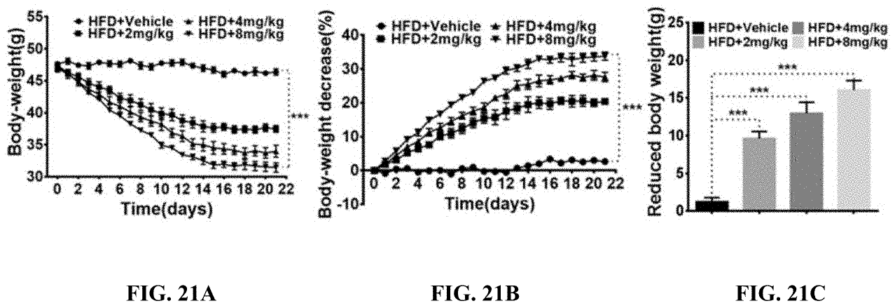

Six-week-old SPF-grade C57BL/6 mice (purchased from Hubei Provincial Center for Disease Control and Prevention) are fed in SPF animal house at a room temperature of 22° C., 4 mice per cage. Animals are fed with 60% calorie high-fat diets (purchased from Bio-medicine) for 14 consecutive weeks. Mice are free to take food and the padding is changed every two days, and the body weights and blood glucose are monitored every week. When the blood glucose is greater than 11 mmol/L and the body weight is greater than 42 g, the administration is started. The drug is dissolved in DMSO solvent and administered by intraperitoneal injection at three doses of 2, 4, and 8 mg/kg for 21 consecutive days. The injection volume is 25 per day. In addition, a vehicle (25 μL DMSO) is established. The food intake and body weight changes of mice in each group are measured every day. On Days 1 and 14, the blood routine and blood biochemical parameters are monitored. At the same time, the glucose tolerance test (GTT) and insulin tolerance test (ITT) are carried out, to determine the body fat parameters of mice on Days 0 and 14. Mice in the blank group and GA-02 treatment group underwent pathological anatomy, to observe the changes in organs before and after administration. Samples are taken to prepare pathological sections. The experimental results are shown in FIGS. 21A-21C.

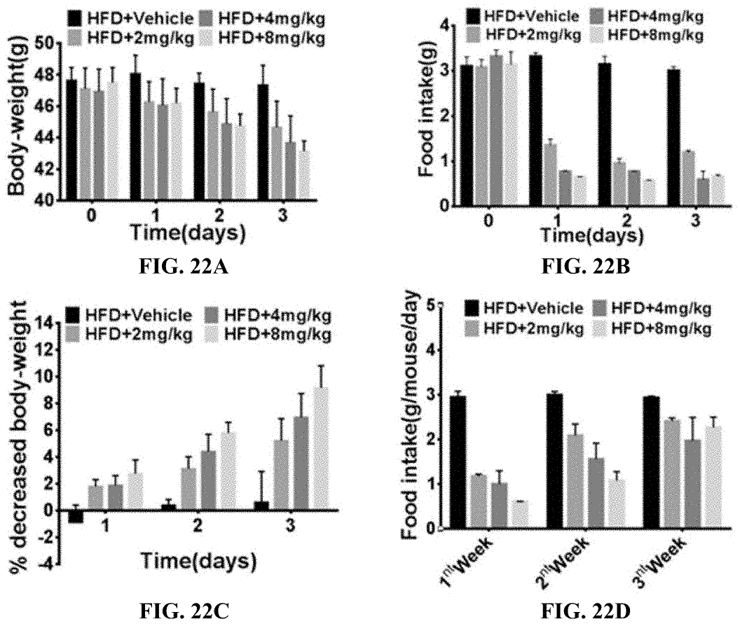

The model is constructed by feeding high-fat diets to C57BL/6 male mice for 14 weeks, as shown in FIGS. 22A-22D, obese model mice with a body weight greater than 42 g and blood glucose level higher than 11 mmol/L. Model mice are grouped by blood glucose and body weight. Model mice in each group are given low-, medium-and high-dose (2, 4, 8 mg/kg, 25 μL/day) GA-03 by intraperitoneal injection for 14 consecutive days, and the mice in the blank control group are given DMSO by intraperitoneal injection (25 μL/day), and the food intakes and body weights are recorded every day. After three weeks of administration, the body weights of the mice in the vehicle control group have no obvious fluctuation, while the body weights of the mice in the low-dose group decrease from 47.11±1.32 g to 37.49±1.28 g, reduced by 20.40±1.89%; the body weights of the medium-dose group decrease from 46.94±1.41 g to 33.99±2.30 g, reduced by 27.63±3.39%; the body weights of the high-dose group decrease from 47.49±0.97 g to 31.45±1.81 g, reduced by 33.80±2.95%. The body weights of mice in each group decrease obviously in a dose-dependent manner.

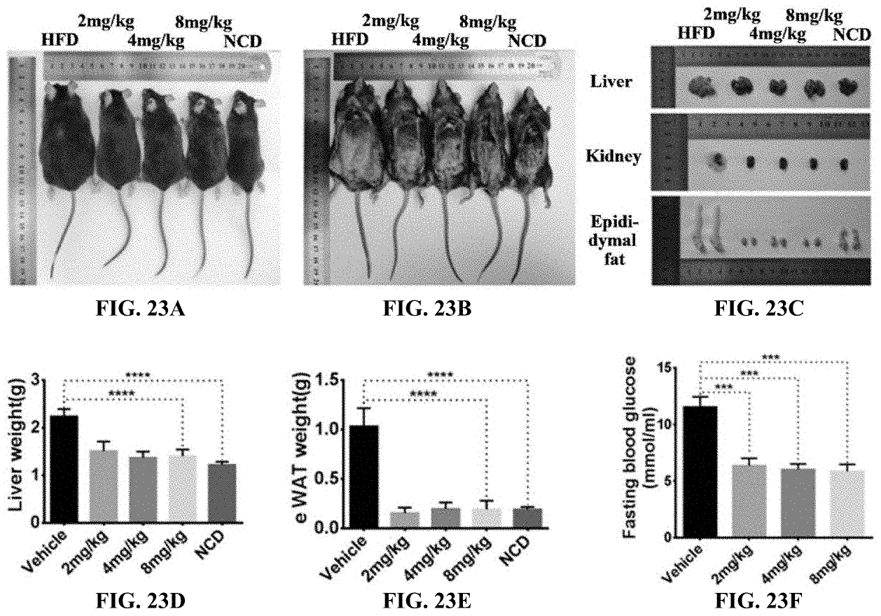

The daily food intakes for the first three days and the first and second weeks of the high-fat-induced obesity mouse models are shown in FIGS. 23A-23F. The results show that, in the high-dose administration group, the average daily food intake on day 0 is 3.14±0.40 g, on the first day of administration, the average daily food intake drops to 0.65±0.02 g and the body weight decreases from 47.49±0.97 g to 46.18±0.95 g; on the second day, the average daily food intake drops to 0.57±0.01 g, and the body weight decreases to 44.74±0.76 g; On the third day, the average daily food intake drops to 0.66±0.06 g, and the body weight decreases to 43.13±0.65 g. The body weight decrease trend in the high-dose group is the same as that in the paired quantitative feeding group, which indirectly indicates that the decrease in body weight is derived from the reduction in the food intakes.

The dissection of mice shows that, the mice after the administration are leaner than the blank vehicle control group, and the large amount of fats in the abdomen decreases obviously after two weeks of administration of GA-03 (FIGS. 23A-23B) in obese mice; the organ anatomy finds a lot of fat in the heart, periphery of the kidneys, epididymis and subcutaneous tissues in obese mice. The livers of the obese group show the symptoms of gray-white fatty liver withy accumulated lipid droplets. After administration, the livers become dark red (FIG. 23C). Compared to the vehicle control group, the weights of liver and epididymal fat of mice in the low-, medium-and high-dose groups decrease in a dose-dependent manner (FIGS. 23D-23E), and the fasting blood glucose levels of mice reduce to a normal level (FIG. 23F).

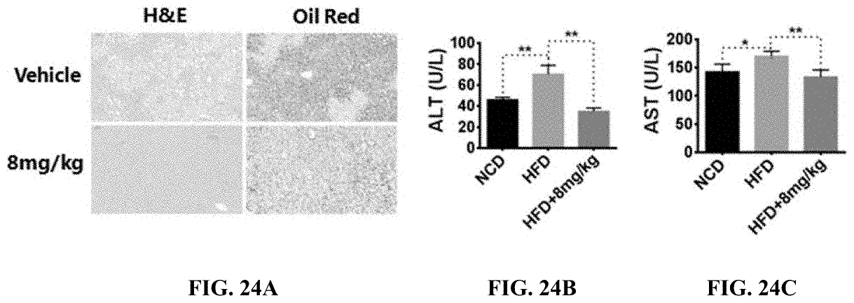

The results of liver oil red O staining and H&E staining sections show there is a large number of lipid droplets in the liver in the blank control group, and the lipid droplets in the liver have completely disappeared in the high-dose GA-03 treatment group (FIG. 24A); the liver function indexes such as AST and ALT are significantly lower than those in the vehicle control group (FIGS. 24B-24C). These results show that the fatty liver symptoms caused by high-fat diets have been well relieved.

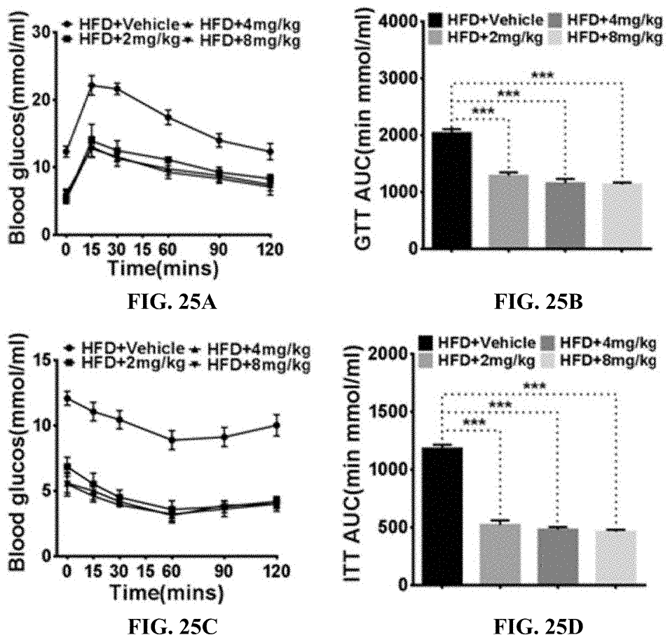

The results of glucose tolerance test (GTT) and insulin tolerance test (ITT) are shown in FIGS. 25A-25D. The overnutrition -induced obesity is often accompanied by symptoms of diabetes such as hyperglycemia and insulin resistance. We perform GTT and ITT on the mice in the high-dose administration group. Results show the blood glucose tolerance and insulin tolerance of mice are significantly improved after GA-03 administration (low-, medium-and high-dose groups), and their fasting blood glucose levels are significantly reduced to normal levels, indicating that the diabetes symptoms caused by obesity have been well recovered (FIGS. 25A-25D).

2) Establishment of a Lean Mouse Model

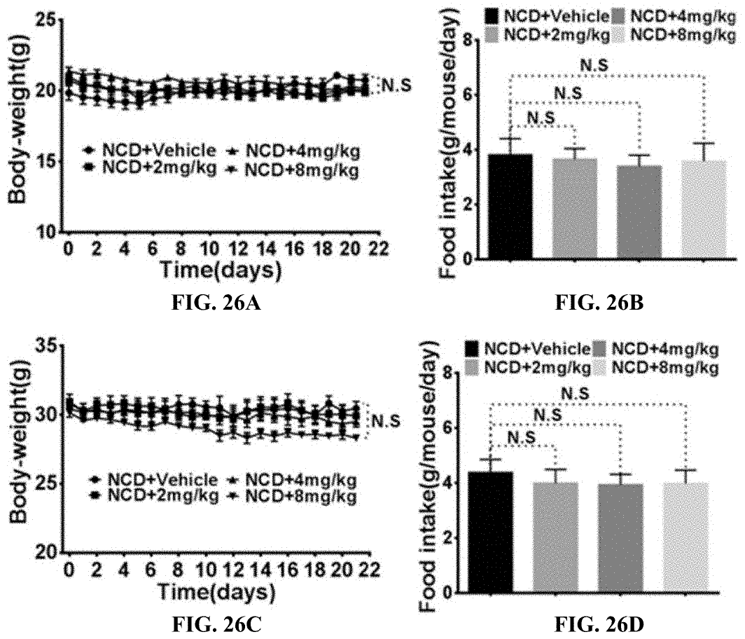

Six-week-old SPF-grade C57BL/6 mice (purchased from Hubei Provincial Center for Disease Control and Prevention) are fed in SPF animal house at a room temperature of 22° C., 4 mice per cage. Animals are fed with common diets (purchased) for 2 consecutive weeks. Mice are free to take food and the padding is changed every two days, and the body weights and blood glucose are monitored every week. When the body weight is greater than 22 g, the administration is started. The drug is dissolved in DMSO solvent and administered by intraperitoneal injection. The injection volume is 25 μL per day. In addition, a vehicle and positive drug control are established. The food intake and body weight changes of mice in each group are measured every day. On Days 1 and 21, the blood routine and blood biochemical parameters are monitored. At the same time, the glucose tolerance test (GTT) and insulin tolerance test (ITT) are carried out. The result is shown in FIGS. 26A-26D.

3) Establishment of a Mildly Obese Mouse Model

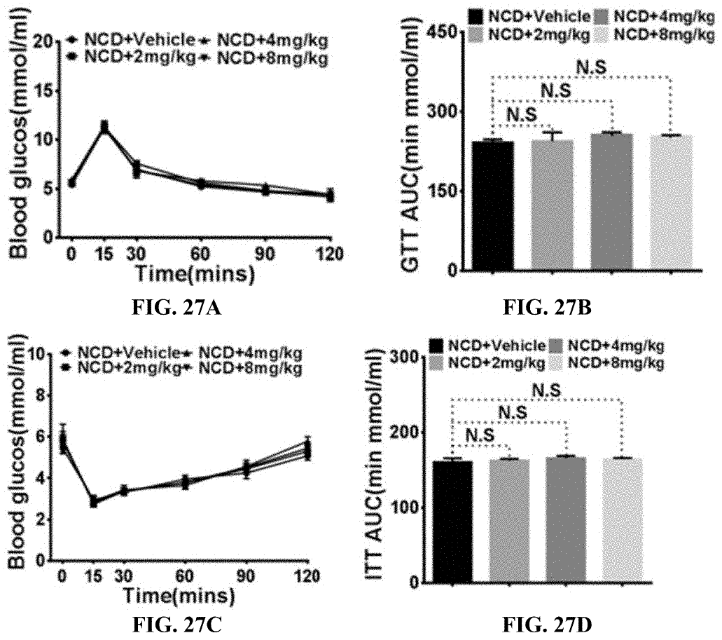

Six-week-old SPF-grade C57BL/6 mice (purchased from Hubei Provincial Center for Disease Control and Prevention) are fed in SPF animal house at a room temperature of 22° C., 4 mice per cage. Animals are fed with common diets (purchased) for 14 consecutive weeks. Mice are free to take food and the padding is changed every two days, and the body weights and blood glucose are monitored every week. When the body weight is greater than 30 g, the administration is started. The drug is dissolved in DMSO solvent and administered by intraperitoneal injection. The injection volume is 25 μL per day. In addition, a vehicle and positive drug control are established. The food intake and body weight changes of mice in each group are measured every day. On Days 1 and 21, the blood routine and blood biochemical parameters are monitored, and the glucose tolerance test (GTT) and insulin tolerance test (ITT) are carried out. The result is shown in FIGS. 27A-27D.

The experimental results show that the intraperitoneal injection of GA-03 has no effect on the food intake and body weight of lean mice, and has obvious effect on appetite suppression and weight reduction of mildly obese mice, and has strong effect on appetite suppression and weight reduction of obese mice.

It will be obvious to those skilled in the art that changes and modifications may be made, and therefore, the aim in the appended claims is to cover all such changes and modifications.

The invention claimed is:

1. A drug design method associated with natural products, the method comprising:
1) acquiring a molecular structure of a to-be-modified natural product with a specific biological activity;
2) using the molecular structure acquired in 1) as a template molecule, selecting a plurality of natural products as a reference molecule set, where the plurality of natural products has the specific biological activity and a structural similarity thereof to the template molecule is within a threshold range; and
3) selecting one or more reference molecules from the reference molecule set obtained in 2), comparing the one or more reference molecules with the template molecule and determining at least one different active functional group therebetween; and constructing the at least one different active functional group on a molecular scaffold shared by the template molecule and the one or more reference molecules, thereby obtaining a modified molecular structure of the natural product with the specific biological activity.

2. The method of claim 1, wherein in 2), the threshold range of the structural similarity of the plurality of natural products with respect to the template molecule is more than 0.2.

3. The method of claim 2, wherein the threshold range of the structural similarity of the plurality of natural products with respect to the template molecule is between 0.2 and 0.9.

4. The method of claim 1, wherein a natural abundance of the plurality of natural products in the reference molecule set in corresponding host plants thereof exceeds a preset bio-content threshold.

5. The method of claim 4, wherein the bio-content threshold is 1%.

6. The method of claim 1, wherein selecting one or more reference molecules from the reference molecule set obtained in 2) comprises: screening one or more reference molecules with the same action target and/or action channel as the template molecule from the reference molecule set.

7. The method of claim 1, wherein determining at least one different active functional group comprises: screening a plurality of active functional groups of the one or more reference molecules through a physiological model or a molecular docking model, and comparing the plurality of active functional groups with the template molecule, to determine at least one different active functional group therebetween.

8. A method for preparing a compound molecule designed according to the drug design method associated with natural products of claim 1, the method comprising:
S1) selecting the template molecule and a reference molecule, selecting one from the template molecule and the reference molecule which has higher bio-content in corresponding host plant species as a raw material; and
S2) constructing the at least one different active functional group between the template molecule and the reference molecule on the raw material through a chemical reaction, thereby obtaining the compound molecule designed by the drug design method.

9. The method of claim 8, wherein the reference molecule is selected as the raw material.

10. The method of claim 8, wherein the specific biological activity is anti-inflammatory activity, and the to-be-modified natural product with the specific biological activity is Celastrol or corosolic acid; and the reference molecule is 18-β glycyrrhetinic acid.

11. The method of claim 10, wherein Celastrol or corosolic acid is modified as follows:
S1) selecting an 18-β glycyrrhetinic acid molecule as the raw material; and
S2) constructing a substituted hydroxyl and a conjugated enone structure on an A ring of Celastrol on the 18-β glycyrrhetic acid molecule; or constructing substituted α, β hydroxyls and a conjugated enone structure on an A ring of corosolic acid on the 18-β glycyrrhetic acid molecule.

12. A pentacyclic triterpenoid compound, comprising a molecular scaffold shared by a template pentacyclic triterpenoid compound and a natural product with a structural-similarity score of 0.2 or more with the template pentacyclic triterpenoid compound, and at least one different active functional group between the template pentacyclic triterpenoid compound and the natural product; wherein the template pentacyclic triterpenoid compound is Celastrol or corosolic acid; the natural product has anti-inflammatory activity, and a natural abundance of the natural product in a host plant thereof exceeds 1%.

13. The compound of claim 12, wherein the natural product is 18-β glycyrrhetinic acid.

14. The compound of claim 12, comprising an oleanane-type triterpene-30 carboxylic acid scaffold and a 3-carbonyl-1,2-enol structure on an A ring thereof.

15. The compound of claim 13, being 3,11-dicarbonyl-1,12-diene-2-hydroxy-oleanane-30 carboxylic acid, or 11-dicarbonyl-12-ene-2α,3β-dihydroxy-oleanane.

16. A method for treatment of chronic inflammation or metabolic syndrome, the method comprising administering to a patient in need thereof the pentacyclic triterpenoid compound of claim 12 or a pharmaceutically acceptable salt thereof.

17. The method of claim 16, wherein the method is used for treatment of obesity or type 2 diabetes.
